(19) United States
(12) Patent Application Publication
(10) Pub. No.: US 2019/0202849 A1
(43) Pub. Date: Jul. 4, 2019

(54) METHOD FOR THE PREPARATION OF SUBSTITUTED THIOLACTONES, NEW SUBSTITUTED THIOLACTONES AND USES THEREOF

(71) Applicants: CENTRE NATIONAL DE LA RECHERCHE SCIENTIFIQUE, PARIS (FR); UNIVERSITE TOULOUSE III - Paul Sabatier, Toulouse Cedex 9 (FR)

(72) Inventors: Olivier COUTELIER, AUZEVILLE TOLOSANE (FR); Mathias DESTARAC, BALMA (FR); Ihor KULAI, KYIV (UA)

(21) Appl. No.: 16/311,551

(22) PCT Filed: Jun. 20, 2017
(86) PCT No.: PCT/FR2017/051637

§ 371 Date: Dec. 19, 2018

(30) Foreign Application Priority Data
Jun. 21, 2016 (FR) 1655781
Nov. 8, 2016 (FR) 1660808

(51) Int. Cl. C07F 9/6553; C08G 77/395

(52) U.S. Cl. CPC C07F 9/655345; C07D 333/32; C08G 77/395

(57) ABSTRACT

The invention relates to a method for preparing substituted thiolactones of formula (I), new substituted thiolactones of formula (I′) that can be obtained by carrying out said method, and the use of substituted thiolactones of formula (I) or (I′) for synthesizing polymers or functionalizing surfaces or polymers.

The present invention relates to the field of thiolactones.

More particularly, the present invention relates to a process for preparing substituted thiolactones of formula (I), novel substituted thiolactones of formula (I′) able to be obtained by carrying out this process, the use of substituted thiolactones of formula (I) or of formula (I′) for the preparation of polymers or for surface functionalization or polymer functionalization.

Thiolactones are heterocyclic compounds analogous to lactones, in which an oxygen atom is replaced by a sulfur atom. The sulfur atom is located in the ring and is adjacent to a carbonyl group. The heterocycle of thiolactones may be substituted by at least one chemical group, in particular by an alkyl or aryl group.

Several processes for synthesizing thiolactones have already been proposed.

F. Korte et al. (Chem. Ber., 1961, 94, 1966), for example, proposed either carrying out thermal cyclization of a mercaptocarboxylic acid bearing an alkyl substituent, or directly substituting a thiolactone with an alkyl radical in the presence of an alkyl halide group (R—X) and lithium dialkylamide (LiNR′₂). These two synthesis routes may be represented by the following reaction scheme (1):

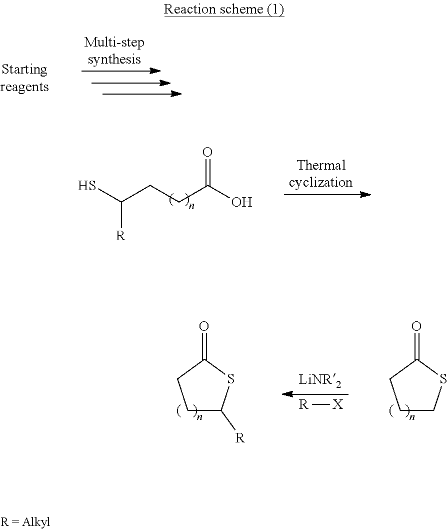

According to this synthesis route, only the final step of thermal cyclization is general, with the preceding steps leading to the mercaptocarboxylic acid and the reagents used being specific to the type of R group that it is desired to introduce to the heterocycle. Moreover, these two synthesis routes do not make it possible to introduce substituents other than alkyl groups.

A more recent synthesis process makes it possible to obtain thiolactones having alkyl or aryl groups (J.-J. Filippi et al., Tet. Lett., 2006, 47, 6067). This process is based on a process of catalytic isomerization of a thionolactone to a thiolactone in the presence of boron trifluoride (BF₃) and diethyl ether (Et₂O) in an organic solvent such as toluene at reflux, according to the following reaction scheme (2):

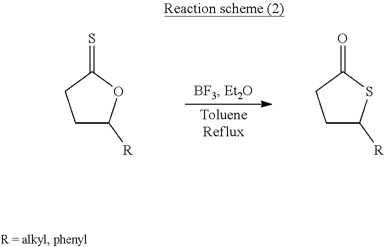

However, this process uses a catalyst of Lewis acid type (boron trifluoride) and cannot be readily used for the synthesis of thiolactones bearing substituents other than alkyl or phenyl groups, such as complex organic functions and/or those that are incompatible with this type of catalyst. Moreover, the isolated thiolactone yields are often low. Finally, this process requires the synthesis of starting thionolactones from the corresponding lactones.

There is therefore a need for a process which makes it possible to synthesize thiolactones substituted by various functional groups in a flexible and simple manner, and according to a process which is both efficient and economical.

Therefore, the first subject of the present invention is a process for preparing substituted thiolactones of the following formula (I):

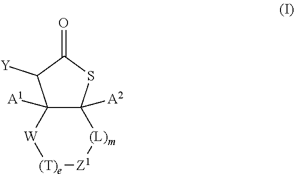

wherein:
A¹ and A², which are identical or different, represent a hydrogen atom or a fluorine atom,
Y represents a hydrogen atom or a group selected from alkyl, hydroxyalkyl, aryl and cyano groups, or a polymer chain;
L is a linker arm,
m is an integer equal to 0 or 1,
T represents CH₂, —O— or —NR⁶—, in which R⁶ represents a hydrogen atom or an alkyl, aryl or aralkyl radical, optionally substituted by a group selected from the groups: maleimide, a group of formula:

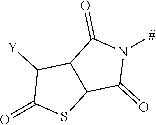

in which the symbol # is the point of attachment of said group to R⁶ and in which Y has the same meaning as that chosen for the radical Y of the formula (I), OH; P(O)(OR⁷)(OR^{7′}) in which the radicals R⁷ and R^{7′}, which are identical or different, represent a hydrogen atom or an alkyl radical; CₙF₂ₙ₊₁ in which n is an integer ranging from 1 to 20; SiR⁸ₚ(OR⁹)₃₋ₚ, in which the radicals R⁸ and R⁹, which are identical or different, represent a hydrogen atom or an alkyl radical and p is an integer equal to 0, 1 or 2; BF₃M⁺, in which M=K or Na; B(OR¹⁰)₂, in which the two radicals R¹⁰, which are identical or different, represent a hydrogen atom, an alkyl radical or form a carbon-based ring with the two oxygen atoms to which they are bonded; OR¹¹, in which R¹¹ represents a hydrogen atom or an alkyl, aryl or aralkyl radical; O(C═O)R¹², in which R¹² represents a hydrogen atom or an alkyl, aryl or aralkyl radical; O(C═O)OR¹³, in which R¹³ represents a hydrogen atom or an alkyl, aryl or aralkyl radical; N⁺R¹⁴R^{14′}R^{14″}A⁻, in which the radicals R¹⁴, R^{14′} and R^{14″}, which are identical or different, represent a hydrogen atom or an alkyl, aryl or aralkyl radical and A represents a chlorine or bromine atom; NR^{15′}(C═O)R¹⁵, in which the radicals R¹⁵ and R^{15′}, which are identical or different, represent a hydrogen atom or an alkyl or aryl radical or are connected together and form a ring such as a pyrrolidone or caprolactam ring; NR^{16′}(C═O)OR¹⁶, in which R¹⁶ et R^{16′}, which are identical or different, represent a hydrogen atom or an alkyl, aryl or aralkyl radical; CN; a halogen atom chosen from Cl, F, and Br; NCS; OCH₂-epoxy; COOR¹⁷, in which R¹⁷ represents a hydrogen atom, an alkyl, aryl or aralkyl radical; CONR¹⁸R^{18′}, in which R¹⁸ and R^{18′}, which are identical or different, represent a hydrogen atom or an alkyl or aryl radical; SO₂R¹⁹, in which R¹⁹ represents an alkyl or aryl radical; azide N₃ and alkyne,
e is an integer equal to 0 or 1,

it being understood that:

1) when A¹=A²=H and e=0, then W represents a hydrogen atom and Z¹ represents a group selected from the groups alkyl; aryl; P(O)(OR⁷)(OR^{7′}), in which the radicals R⁷ and R^{7′}, which are identical or different, represent a hydrogen atom or an alkyl radical; CₙF₂ₙ₊₁ in which n is an integer ranging from 1 to 20; SiR⁸ₚ(OR⁹)₃₋ₚ, in which the radicals R⁸ and R⁹, which are identical or different, represent a hydrogen atom or an alkyl radical and p is an integer equal to 0, 1 or 2; BF₃M⁺, in which M=K or Na; B(OR¹⁰)₂, in which the two radicals R¹⁰, which are identical or different, represent a hydrogen atom, an alkyl radical or form a carbon-based ring with the two oxygen atoms to which they are bonded; OR¹¹, in which R¹¹ represents a hydrogen atom or an alkyl, aryl or aralkyl radical; O(C═O)R¹², in which R¹² represents a hydrogen atom or an alkyl, aryl or aralkyl radical; O(C═O)OR¹³, in which R¹³ represents a hydrogen atom or an alkyl, aryl or aralkyl radical; N⁺R¹⁴R^{14′}R^{14″}A⁻, in which the radicals R¹⁴, R^{14′} and R^{14″}, which are identical or different, represent a hydrogen atom or an alkyl, aryl or aralkyl radical and A represents a chlorine or bromine atom; NR^{15′}(C═O)R¹⁵, in which the radicals R¹⁵ and R^{15′}, which are identical or different, represent a hydrogen atom or an alkyl or aryl radical or are connected together and form a ring such as a pyrrolidone or caprolactam ring; NR^{16′}(C═O)OR¹⁶, in which R¹⁶ and R^{16′}, which are identical or different, represent a hydrogen atom or an alkyl, aryl or aralkyl radical; CN; a halogen atom chosen from Cl, F, and Br; NCS; OCH₂-epoxy; COOR¹⁷, in which R¹⁷ represents a hydrogen atom, an alkyl, aryl or aralkyl radical; CONR¹⁸R^{18′}, in which R¹⁸ and R^{18′}, which are identical or different, represent a hydrogen atom or an alkyl or aryl radical; SO₂R¹⁹, in which R¹⁹ represents an alkyl or aryl radical; azide N₃ and alkyne; and a thiolactone ring of formula

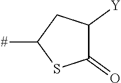

in which the symbol # is the point of attachment of the thiolactone ring to L and in which Y has the same meaning as that chosen for the radical Y of the formula (I),

2) when A¹=A²=H, m=1 and e=0, then W represents a hydrogen atom and Z¹ may also represent a hydrogen atom;

3) when A¹=A²=H, e=1 and m=0, then Z¹ and W are identical and represent —CH₂— or CO;

4) when A¹=A²=H, e=1, m=1 and T=CH₂ then Z¹=W=—CH₂,

5) when A¹=A²=F, e=0 and m=0, then W represents a fluorine atom and Z¹=F or represents a linear or branched chain OC_{q}F_{2q+1} in which q is an integer ranging from 1 to 5, [OCF₂CF(CF₃)]ᵣOC₃F₇, with r=an integer ranging from 0 to 20, OC₂F₄SO₂F, OCF₂CF(CF₃)OC₂F₄SO₂F, or OCF₂CF(CF₃)OC₂F₄CO₂CH₃;

6) when A¹=A²=F, e=1 and m=0, then W=Z¹=CF₂ and T=(CF₂)ₛ, with s=an integer ranging from 1 to 5; and

7) when A¹=H, A²=F, and e=m=0, then W represents a hydrogen atom and Z¹=F or CₙF₂ₙ₊₁, in which n is an integer ranging from 1 to 20;

said process being characterized in that it comprises at least the following steps:

1) a step during which, in the presence of a radical initiator, a xanthate of the following formula (II) is reacted:

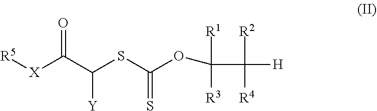

wherein:
R¹, R², R³ and R⁴, which are identical or different, represent a hydrogen atom or a group chosen from saturated or unsaturated heterocycloalkyl, alkyl, acyl, aryl, alkene, alkyne, cycloalkyl, or heterocycloaryl groups and polymer chains, it being understood that the radicals R¹, R², R³ and R⁴ may also form, together, a saturated, unsaturated or aromatic cycloalkyl or heterocycloalkyl group; it being understood that at least one of the radicals R¹ and R³ is other than a hydrogen atom;
Y has the same meaning as in the formula (I) above,
X represents NR²⁰, in which R²⁰ represents a hydrogen atom or an alkyl radical or —O—,
R⁵ is chosen from a saturated, unsaturated or aromatic heterocycloalkyl, alkyl, acyl, aryl, aralkyl or cycloalkyl group;

with a monomer comprising at least one ethylenic unsaturation of the following formula (III):

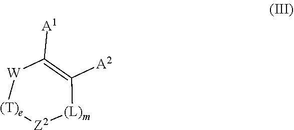

wherein:
A¹ and A², which are identical or different, represent a hydrogen atom or a fluorine atom,
L, m, T and e have the same meaning as in the formula (I) above;

it being understood that:

1) when A¹=A²=H and e=0, then W represents a hydrogen atom and Z² represents a group selected from the groups alkyl; aryl; P(O)(OR⁷)(OR^{7′}), in which the radicals R⁷ and R^{7′}, which are identical or different, represent a hydrogen atom or an alkyl radical; CₙF₂ₙ₊₁, in which n is an integer ranging from 1 to 20; SiR⁸ₚ(OR⁹)₃₋ₚ, in which the radicals R⁸ and R⁹, which are identical or different, represent a hydrogen atom or an alkyl radical and p is an integer equal to 0, 1 or 2; BF₃M⁺, in which M=K or Na; B(OR¹⁰)₂, in which the two radicals R¹⁰, which are identical or different, represent a hydrogen atom, an alkyl radical or form a carbon-based ring with the two oxygen atoms to which they are bonded; OR¹¹, in which R¹¹ represents a hydrogen atom or an alkyl, aryl or aralkyl radical; O(C═O)R¹², in which R¹² represents a hydrogen atom or an alkyl, aryl or aralkyl radical; O(C═O)OR¹³, in which R¹³ represents a hydrogen atom or an alkyl, aryl or aralkyl radical; N⁺R¹⁴R^{14′}R^{14″}A⁻, in which the radicals R¹⁴, R^{14′} and R^{14″}, which are identical or different, represent a hydrogen atom or an alkyl, aryl or aralkyl radical and A represents a chlorine or bromine atom; NR^{15′}(C═O)R¹⁵, in which the radicals R¹⁵ and R^{15′}, which are identical or different, represent a hydrogen atom or an alkyl or aryl radical or are connected together and form a ring such as a pyrrolidone or caprolactam ring; NR^{16′}(C═O)OR¹⁶, in which R¹⁶ and R^{16′}, which are identical or different, represent a hydrogen atom or an alkyl, aryl or aralkyl radical; CN; a halogen atom chosen from Cl, F, and Br; NCS; OCH₂-epoxy; COOR¹⁷, in which R¹⁷ represents a hydrogen atom, an alkyl, aryl or aralkyl radical; CONR¹⁸R^{18′}, in which R¹⁸ and R^{18′}, which are identical or different, represent a hydrogen atom or an alkyl or aryl radical; SO₂R¹⁹, in which R¹⁹ represents an alkyl or aryl radical; azide N₃, alkyne and C₂H₃ (i.e. CH═CH₂);

2) when A¹=A²=H, m=1 and e=0, then W represents a hydrogen atom and Z² may also represent a hydrogen atom,

3) when A¹=A²=H, e=1 and m=0, then Z² and W are identical and represent —CH₂— or CO;

4) when A¹=A²=H, e=1, m=1 and T=CH₂, then Z²=W=—CH₂,

5) when A¹=A²=F, e=0 and m=0, then W represents a fluorine atom and Z²=F or represents a linear or branched chain OC_{q}F_{2q+1} in which q is an integer ranging from 1 to 5, [OCF₂CF(CF₃)]ᵣOC₃F₇, with r=an integer ranging from 0 to 20, OC₂F₄SO₂F, OCF₂CF(CF₃)OC₂F₄SO₂F, or OCF₂CF(CF₃)OC₂F₄CO₂CH₃; and

6) when A¹=A²=F, e=1 and m=0, then W=Z²=CF₂ and T=(CF₂)ₛ, with s=an integer ranging from 1 to 5;

7) when A¹=H, A²=F, and e=m=0, then W represents a hydrogen atom and Z²=F or CₙH₂ₙ₊₁, in which n is an integer ranging from 1 to 20;

to form a monoadduct of the following formula (IV):

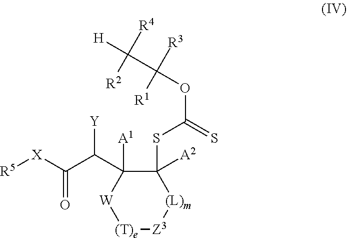

wherein:
A¹ and A², which are identical or different, represent a hydrogen atom or a fluorine atom,
R¹, R², R³, R⁴, R⁵, X and Y have the same meaning as in the formula (II) above,
L, m, T and e have the same meaning as in the formula (I) above,

it being understood that:

1) when A¹=A²=H and e=0, then W represents a hydrogen atom and Z³ represents a group selected from the groups alkyl; aryl; P(O)(OR⁷)(OR^{7′}), in which the radicals R⁷ and R^{7′}, which are identical or different, represent a hydrogen atom or an alkyl radical; CₙF₂ₙ₊₁, in which n is an integer ranging from 1 to 20; SiR⁸ₚ(OR⁹)₃₋ₚ, in which the radicals R⁸ and R⁹, which are identical or different, represent a hydrogen atom or an alkyl radical and p is an integer equal to 0, 1 or 2; BF₃M⁺, in which M=K or Na; B(OR¹⁰)₂, in which the two radicals R¹⁰, which are identical or different, represent a hydrogen atom, an alkyl radical or form a carbon-based ring with the two oxygen atoms to which they are bonded; OR¹¹, in which R¹¹ represents a hydrogen atom or an alkyl, aryl or aralkyl radical; O(C═O)R¹², in which R¹² represents a hydrogen atom or an alkyl, aryl or aralkyl radical; O(C═O)OR¹³, in which R¹³ represents a hydrogen atom or an alkyl, aryl or aralkyl radical; N⁺R¹⁴R^{14′}R^{14″}A⁻, in which the radicals R¹⁴, R^{14′} and R^{14″}, which are identical or different, represent a hydrogen atom or an alkyl, aryl or aralkyl radical and A represents a chlorine or bromine atom; NR^{15′}(C═O)R¹⁵, in which the radicals R¹⁵ and R^{15′}, which are identical or different, represent a hydrogen atom or an alkyl or aryl radical or are connected together and form a ring such as a pyrrolidone or caprolactam ring; NR^{16′}(C═O)OR¹⁶, in which R¹⁶ and R^{16′}, which are identical or different, represent a hydrogen atom or an alkyl, aryl or aralkyl radical; CN; a halogen atom chosen from Cl, F, and Br; NCS; OCH₂-epoxy; COOR¹⁷, in which R¹⁷ represents a hydrogen atom, an alkyl, aryl or aralkyl radical; CONR¹⁸R^{18′}, in which R¹⁸ and R^{18′}, which are identical or different, represent a hydrogen atom or an alkyl or aryl radical; SO₂R¹⁹, in which R¹⁹ represents an alkyl or aryl radical; azide N₃, alkyne, and a group of the following formula (V):

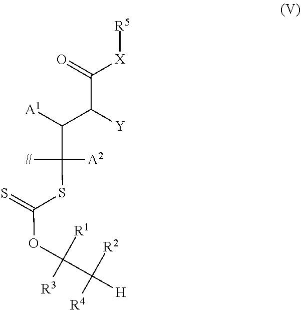

in which the symbol # is the point of attachment of the group of formula (V) to L and R¹, R², R³, R⁴, R⁵ and Y respectively have the same meaning as that chosen for R¹, R², R³, R⁴, R⁵ and Y in the formula (IV),

2) when A¹=A²=H, m=1 and e=0, then W represents a hydrogen atom and Z³ may also represent a hydrogen atom,

3) when A¹=A²=H, e=1 and m=0, then Z³ and W are identical and represent —CH₂— or CO;

4) when A¹=A²=H, e=1, m=1 and T=CH₂, then Z³=W=—CH₂,

5) when A¹=A²=F, e=0 and m=0, then W represents a fluorine atom and Z³=F or represents a linear or branched chain OC_{q}F_{2q+1} in which q is an integer ranging from 1 to 5, [OCF₂CF(CF₃)]ᵣOC₃F₇, with r=an integer ranging from 0 to 20, OC₂F₄SO₂F, OCF₂CF(CF₃)OC₂F₄SO₂F, or OCF₂CF(CF₃)OC₂F₄CO₂CH₃; and

6) when A¹=A²=F, e=1 and m=0, then W=Z³=CF₂ and T=(CF₂)ₛ, with s=an integer ranging from 1 to 5;

7) when A¹=H, A²=F, and e=m=0, then W represents a hydrogen atom and Z³=F or CₙF₂ₙ₊₁, in which n is an integer ranging from 1 to 20;

then

2) a step of thermolysis of the monoadduct of formula (IV) obtained above in the preceding step, to form a corresponding substituted thiolactone of formula (I).

The process for preparing the substituted thiolactones of formula (I) in accordance with the invention may be represented by the following reaction scheme (3):

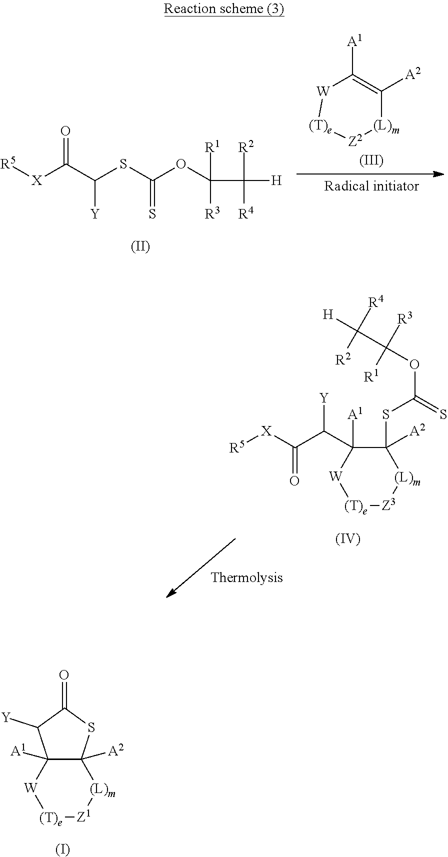

By virtue of the process in accordance with the present invention, and as described above, it is now possible to simply and quickly, and with a good yield, obtain thiolactones substituted by various organic groups.

According to a first particularly advantageous embodiment form, the process of the invention leads to the formation of a dithiolactone of formula (Ia), in which A¹, A², L, m and Y have the same meaning as in formula (I) and W=H, said dithiolactone being obtained according to the reaction scheme (4a)

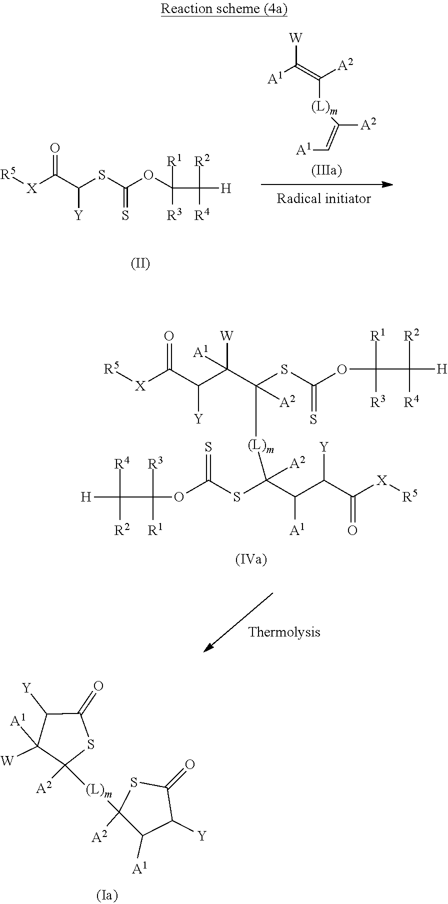

in which
the monomer comprising an ethylenic unsaturation of general formula (IIIa) corresponds to a monomer of formula (III) in which e=0, Z²=C₂H₃ (i.e. CH═CH₂) and W=H and A¹, A², L and m having the same meaning as in formula (III),
the monoadduct of general formula (IVa) corresponds to a monoadduct of formula (IV) in which e=0, Z³ is a group of formula (V) as defined above, in which W=H and A¹, A², L, m, X, R¹, R², R³, R⁴ and R⁵ have the same meaning as in formula (IV).

According to another particularly advantageous embodiment form, the process of the invention leads to the formation of a dithiolactone of formula (Ib), in which A¹, A² and Y have the same definition as in formula (I), m=0, W and Z¹=CO, e=1, T=NR⁶, in which R⁶ has the same meaning as that chosen for the radical R⁶ of formula (I), said dithiolactone being obtained according to the following reaction scheme (4b):

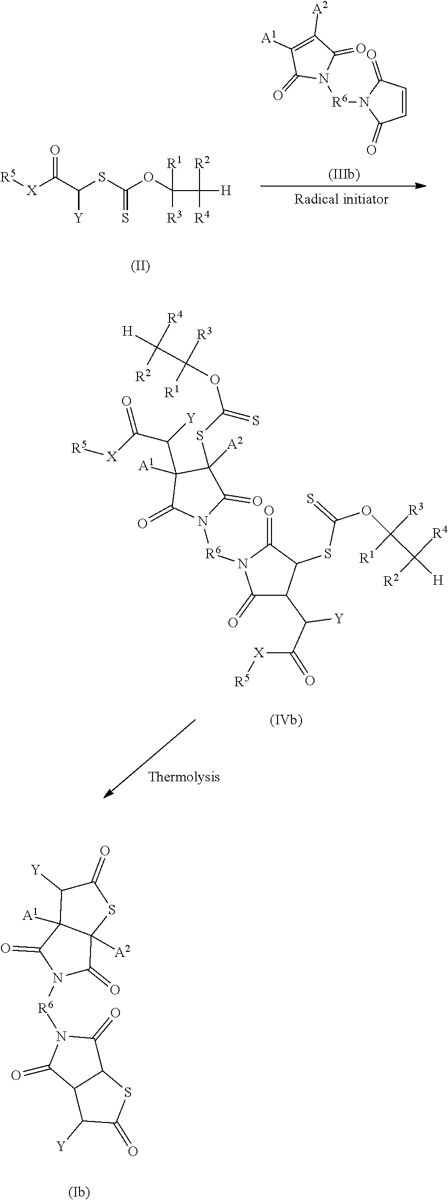

in which the monomer comprising an ethylenic unsaturation of general formula (IIIb) corresponds to a monomer of formula (III) in which A¹ and A² have the same meaning as in formula (I), m=0, Z²=W=CO, e=1 and T=NR⁶ with R⁶=the same meaning as that chosen for the radical R⁶ of formula (I), and
the monoadduct of general formula (IVb) corresponds to a monoadduct of formula (IV) in which m=0, Z³=W=CO, e=1 and T=NR⁶, with R⁶=an alkyl, aryl, or aralkyl radical substituted by a maleimide group, and A¹, A², Y, X, R¹, R², R³, R⁴ and R⁵ have the same definition as in formula (I).

According to the invention, the thiolactones of formula (I) in which A¹ and A² represent a hydrogen atom are preferred.

The nature of the linker arm L is not critical. The linker arm L may especially be an optionally fluorinated or perfluorinated hydrocarbon-based chain, in particular optionally fluorinated or perfluorinated linear alkylene, which may be interrupted by one or more heteroatoms, preferably by one or more oxygen atoms and preferentially at least one oxygen atom is in the penultimate position, said optionally fluorinated or perfluorinated hydrocarbon-based chain having from 1 to 100 carbon atoms, preferentially from 1 to 12 carbon atoms, and even more preferentially from 1 to 3 carbon atoms.

According to a particularly preferred embodiment form of the invention, the linker arm L is a linear alkylene chain having from 1 to 8 carbon atoms.

The alkyl radicals mentioned for R¹, R², R³, R⁴, R⁵, R⁶, Y and the radicals Z (Z¹, Z² and Z³) may be linear, branched, substituted or unsubstituted and comprise from 1 to 12 carbon atoms. They are preferably chosen from methyl, ethyl, n-propyl, iso-propyl, n-butyl, 2-butyl, iso-butyl, tert-butyl, n-pentyl, iso-pentyl, neo-pentyl, tert-pentyl, hexyl, n-octyl, iso-octyl, 2-ethyl-1-hexyl, 2,2,4-trimethylpentyl, nonyl, decyl, dodecyl and benzyl radicals. Among such radicals, methyl, ethyl, n-propyl, iso-propyl and n-butyl radicals are most particularly preferred.

For the purposes of the present invention, an acyl group denotes a group of formula —C(═O)-D, in which D denotes a hydrogen atom or a linear or branched, saturated or unsaturated hydrocarbon-based chain comprising from 1 to 12 carbon atoms. Among such acyl groups mentioned for R¹, R², R³, R⁴, R⁵, R⁶, Z¹, Z² and Z³, mention may especially be made of formyl, acetyl, propanoyl pivaloyl groups.

For the purposes of the invention, aryl group is intended to mean a monocyclic or polycyclic aromatic hydrocarbon-based group that is optionally monosubstituted or polysubstituted. By way of aryl radical mentioned for R¹, R², R³, R⁴, R⁵, R⁶, Z¹, Z² and Z³, mention may in particular be made of naphthyl, anthranyl, phenanthryl, o-tolyl, p-tolyl, xylyl, ethylphenyl, mesityl and phenyl groups. Among such groups, the phenyl group is particularly preferred.

For the purposes of the present invention, the cycloalkyl group is a saturated cyclic group comprising from 3 to 10 carbon atoms. Among such cycloalkyl groups mentioned for R¹, R², R³, R⁴, R⁵ and R⁶, mention may in particular be made of cyclopropyl, cyclobutyl, cyclopentyl, cyclohexyl and cycloheptyl groups.

Still for the purposes of the present invention, a heterocycloalkyl group is a saturated cyclic group comprising from 3 to 9 carbon atoms and at least one heteroatom chosen from N, O, P, Si and S. Among such heterocycloalkyl groups mentioned for R¹, R², R³, R⁴, R⁵ and R⁶, mention may in particular be made of oxacyclopropanyl, azacyclopropanyl, thiacyclopropanyl, tetrahydrofuranyl, pyrrolidinyl, tetrahydrothiophenyl, tetrahydropyranyl, piperidinyl, piperazinyl and thiacyclohexane groups.

For the purposes of the present invention, a heterocycloaryl group is a monocyclic or polycyclic aromatic group comprising from 5 to 6 carbon atoms and at least one heteroatom chosen from N, O, P, Si and S. Among such heterocycloaryl groups mentioned for R¹, R², R³, R⁴, R⁵ and R⁶, mention may in particular be made of furanyl, thiophenyl, pyrrolyl, pyridinyl, pyranyl, oxazinyl, thazinyl, pyrimidinyl, piperazinyl and thiinyl groups.

In a particular embodiment, R¹, R², R³ and R⁴, which are identical or different, represent a hydrogen atom or an alkyl group, of course with the proviso, as indicated above, that at least one of the groups R¹ and R³ is other than a hydrogen atom.

According to a particularly preferred embodiment form of the invention, R¹ (respectively R³) is an alkyl group, especially a methyl group, and R³ (respectively R¹) is a hydrogen atom.

Preferably, at least one of the groups R² and R⁴ is other than a hydrogen atom.

Further preferably, R² (respectively R⁴) is an alkyl group, especially a methyl group, and R⁴ (respectively R²) is a hydrogen atom or an alkyl group, especially a methyl group.

Finally, according to the present invention, polymer chain is intended to mean any linked sequence of monomer units obtained by a radical polymerization process controlled by reversible addition-fragmentation (RAFT/MADIX process as described, for example, by Moad, G. et al., Aust. J. Chem., 2012, 65(8), 985-1076) or atom transfer (ATRP process, i.e. Atom Transfer Radical Polymerization as described, for example, by Matyjaszewski, K., Chem. Rev., 2001, 101(9), 2921-2990), carried out such that the terminal monomer unit connected respectively to the sulfur atom of the thiocarbonylthio group (RAFT/MADIX), or halogen (Cl, Br) group for ATRP, is of acrylate type, for example methyl acrylate, or acrylamido, such as N-isopropylacrylamide.

For the purposes of the present invention, in the compounds of formula (I), (III) or (IIIa) and (IV) or (IVa), when e=0 then T is absent and W and, respectively, Z¹, Z² and Z³ are not connected.

The process in accordance with the invention is preferably carried out for preparing substituted thiolactones of formula (I) as defined above, in which:
Z¹ is preferably a group chosen from the groups P(O)(OR⁷)(OR^{7′}), in particular a dimethylphosphonate group (R⁷=R^{7′}=—CH₃) or diethylphosphonate group (R⁷=R^{7′}=—CH₂CH₃); CₙF₂ₙ₊₁ in which n is preferably an integer ranging from 1 to 10; B(OR¹⁰)₂; OR¹¹; SiR⁸ₚ(OR⁹)₃₋ₚ; NR^{15′}(C═O)R¹⁵, in which R^{15′} is a hydrogen atom and NR^{16′}(C═O)OR¹⁶, in which R^{16′} is a hydrogen atom, and/or
Y is preferably a hydrogen atom or a group chosen from an alkyl radical, in particular methyl and hydroxymethyl.

According to a particularly preferred embodiment form of the invention, R⁵ is an alkyl group, especially a methyl group.

The process in accordance with the invention is preferably carried out to prepare thiolactones of formula (I) in which e=0, m=1 and Z¹ is other than a hydrogen atom.

According to a particularly advantageous embodiment form, the process of the invention leads to the formation of a thiolactone of formula (I) chosen from:
dimethyl 5-oxo-tetrahydrothiophen-2-ylphosphonate (TL1);
diethyl (4-methyl-5-oxo-tetrahydrothiophen-2-yl)methylphosphonate (TL2);
diethyl (5-oxo-tetrahydrothiophen-2-yl)methylphosphonate (TL3);
3-methyl-5-pentyl-dihydrothiophen-2(3H)-one (TL4);
5-pentyl-dihydrothiophen-2(3H)-one (TL5);
3-methyl-5-(perfluorooctyl)dihydrothiophen-2(3H)-one (TL6);
3-methyl-5-phenyldihydro-2H-thieno[2,3-c]pyrrole-2,4,6(3H,5H)-trione (TL7);
3-methyl-5-(perfluorobutyl)dihydrothiophen-2(3H)-one (TL8);
(4-methyl-5-oxo-tetrahydrothiophen-2-yl)phosphonic acid (TL9);
((4-methyl-5-oxo-tetrahydrothiophen-2-yl)methyl)phosphonic acid (TL10);
(5-oxo-tetrahydrothiophen-2-yl)methylphosphonic acid (TL11);
3-methyl-5-(trimethoxysilyl)dihydrothiophen-2(3H)-one (TL12);
5-(trimethoxysilyl)dihydrothiophen-2(3H)-one (TL13);
tert-butyl-N-(4-methyl-5-oxo-tetrahydrothiophen-2-yl)carbamate (TL14);
tert-butyl (5-oxotetrahydrothiophen-2-yl)carbamate (TL15);
3-methyl-5-(oxiran-2-ylmethoxy)dihydrothiophen-2(2H)-one (TL16);
5-((oxiran-2-yloxy)methyl)dihydrothiophen-2(3H)-one (TL17);
3-methyl-5-(4,4,5,5-tetramethyl-1,3,2-dioxaborolan-2-yl)dihydrothiophen-2(3H)-one (TL18);
(5-oxo-tetrahydrothiophen-2-yl)phosphonic acid (TL19);
5-(4,4,5,5-tetramethyl-1,3,2-dioxaborolan-2-yl)dihydrothiophen-2(3H)-one (TL20);
5-(perfluorooctyl)dihydrothiophen-2(3H)-one (TL21);
5-(perfluorobutyl)dihydrothiophen-2(3H)-one (TL22);
dihydro-5-(3-(tetrahydro-4-methyl-5-oxothiophen-2-yl)propyl)-3-methylthiophen-2(3H)-one (TL23);
5-(9-hydroxynonyl)-3-methyldihydrothiophen-2(3H)-one (TL24);
5-(9-bromononyl)3-methyldihydrothiophen-2(3H)-one (TL25);
5,5′-(ethane-1,2-diyl)bis(3-methyldihydrothiophen-2(3H)-one (TL26), and
5,5′-(hexane-1,6-diyl)bis(3-methyldihydrothiophen-2(3H)-one (TL27).

When they are not commercially available, the xanthates of formula (II) may be obtained according to a process analogous to that used in international application WO 2004/024681 and consisting in:

a) in a first step, reacting, in an organic solvent, an alcohol of following formula (VI):

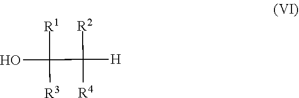

in which the radicals R¹, R², R³ and R⁴ have the same meaning as in the compounds of formula (II) above

with carbon disulfide in the presence of a base to obtain a salt of following formula (VII):

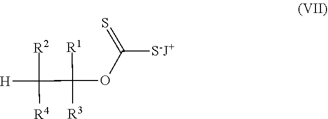

in which the radicals R¹, R², R³ and R⁴ have the same meaning as in the xanthate of formula (II) above, and J⁺ is a cation chosen from alkali metals, such as a K⁺ or Na⁺ cation, then

b) in a second step, reacting the compound of formula (VII) obtained above in the preceding step with a compound of following formula (VIII):

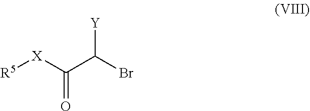

in which R⁵ and X have the same meaning as in the xanthate of formula (II) above, to obtain a corresponding xanthate of formula (II).

The first step a) of preparing a compound of formula (VII) is preferably carried out at room temperature in an organic solvent such as tetrahydrofuran and using a strong base, preferably potassium hydroxide. The duration of step a) is generally from approximately 20 to 24 hours.

The second step b) of preparing a xanthate of formula (II) is preferably carried out in an organic solvent such as acetone and in an ice bath, since the reaction of addition of the compound of formula (VIII) is highly exothermic.

Once the addition of the compound of formula (VIII) has finished, the reaction is preferably carried out at room temperature for a duration of approximately 2 to 4 hours. When the reaction has finished, the xanthate of formula (II) thus obtained is filtered, then the filtrate is preferably concentrated under vacuum. The xanthate of formula (II) may subsequently be used in the process in accordance with the present invention without additional purification.

According to a first embodiment of the invention, the xanthate of formula (II) is O-1,2-dimethylpropyl S-methoxycarbonylmethyl xanthate (XA1) or O-1,2-dimethylpropyl S-(1-methoxycarbonyl)ethyl xanthate (XA2).

According to a second embodiment of the invention, the xanthate of formula (II) is as defined in the invention [i.e. with R¹, R², R³, R⁴, Y, X and R⁵ as defined in the invention], excluding the xanthates of the first embodiment above.

Step 1) of preparing the monoadduct of formula (IV) of the process in accordance with the invention may be carried out without solvent, in water or in an organic solvent. It is preferably carried out in an organic solvent or in water, and even more preferentially in an organic solvent. The organic solvent of use during this step 1) is thus preferably chosen from toluene, tetrahydrofuran (THF), ethyl acetate and 1,4-dioxane. Among such organic solvents, toluene is particularly preferred.

For the purposes of the present invention, radical initiator is intended to mean a chemical entity capable of forming free radicals, that is to say a chemical entity having one or more unpaired electrons in its outer shell.

According to the process in accordance with the invention, the radical initiator used during step 1) is preferably chosen from organic peroxides, azo derivatives, redox couples that generate radicals and redox systems.

Among the organic peroxides, mention may in particular be made of dilauroyl peroxide (LPO), t-butyl peroxyacetate, t-butyl peroxybenzoate, t-butyl peroxyoctoate, t-butyl peroxydodecanoate, t-butyl peroxyisobutyrate, t-amyl peroxypivalate, t-butyl peroxypivalate, diisopropyl peroxydicarbonate, dicyclohexyl peroxydicarbonate, dicumyl peroxide, dibenzoyl peroxide, potassium peroxydisulfate, sodium peroxydisulfate and ammonium peroxydisulfate. Among these organic peroxides, LPO is particularly preferred.

Among the azo derivatives, mention may in particular be made of 2,2′-azobis(isobutyronitrile), 2,2′-azobis(2-cyano-2-butane), dimethyl-2,2′-azobisdimethylisobutyrate, 4,4′-azobis-(4-cyanopentanoic acid), 1,1′-azobis(cyclohexanecarbonitrile), 2-(t-butylazo)-2-cyanopropane, 2,2′-azobis[2-methyl-N(1,1)-bis(hydroxymethyl)-2-hydroxyethyl] propanamide, 2,2′-azobis[2-methyl-N-hydroxyethyl] propanamide, 2,2′-azobis([N,N′-dimethyleneisobutyramidine) dihydrochloride, 2,2′-azobis(2-amidinopropane) dihydrochloride, 2,2′-azobis(N,N′-dimethylene isobutyramine), 2,2′-azobis(2-methyl-N-[1, bis(hydroxymethyl)-2-hydroxyethyl]propionamide), 2,2′-azobis(2-methyl-N-[1,1-bis(hydroxymethyl)propionamide], 2,2′-azobis[2-methyl-N-(2-hydroxyethyl)propionamide], 2,2′-azobis(isobutyramide) dihydrate, 2,2′-azobis(2,2,4-trimethylpentane) and 2,2′-azobis(2-methylpropane).

The redox systems are for example chosen from systems comprising combinations such as:
mixtures of hydrogen peroxide, an alkyl, a perester, a percarbonate and similar compounds and an iron salt, a titanium salt, zinc formaldehyde sulfoxylate or sodium formaldehyde sulfoxylate, and a reducing sugar,
mixtures of an alkali metal or ammonium persulfate, perborate or perchlorate and an alkali metal bisulfite, such as sodium metabisulfite, and a reducing sugar, and
mixtures of an alkali metal persulfate with an arylphosphinic acid, such as benzenephosphonic acid and other similar compounds, and a reducing sugar.

Among such redox systems, the combination of ammonium persulfate and sodium formaldehyde sulfoxylate is most particularly preferred.

Moreover, during step 1), the radical initiator may be added to the reaction medium all at once or in several goes, that is to say portionwise. According to a preferred embodiment form of the process of the invention, the radical initiator is added to the reaction medium portionwise.

According to a first embodiment of the invention, the monoadduct of formula (IV) as obtained at the end of step 1) is chosen from O-1,2-dimethylpropyl S-(1-(dimethylphosphoryl)-3-(methoxycarbonyl))propyl xanthate (XA1VP), O-1,2-dimethylpropyl S-(1-(diethylphosphoryl)-4-(methoxycarbonyl))pent-2-yl xanthate (XA2AP), O-1,2-dimethylpropyl S-(1-(diethylphosphoryl)-4-(methoxycarbonyl))but-2-yl xanthate (XA3AP), O-1,2-dimethylpropyl S-2-(methoxycarbonyl)non-4-yl xanthate (XA4H), O-1,2-dimethylpropyl S-1-(methoxycarbonyl)oct-3-yl xanthate (XA5H), methyl-5,5,6,6,7,7,8,8,9,9,10,10,11,11,12,12,12-heptadecafluoro-2-methyl-4-((((3-methylbutan-2-yl)oxy)carbonothioyl)thio)dodecanoate (XA6DF), the monoadduct of example 7 below (XA7MAL) and methyl 5,5,6,6,7,7,8,8,8-nonafluoro-2-methyl-4-((((3-methylbutan-2-yl)oxy)carbonothioyl)thio)octanoate (XA8HF).

According to a second embodiment of the invention, the monoadduct of formula (IV) is as defined in the invention [i.e. with R¹, R², R³, R⁴, Y, X, R⁵, Z³, W, T, e, L and m as defined in the invention], excluding the monoadducts of the first embodiment above.

The monomers of formula (III) are preferably chosen from compounds which are monomers that are only slightly, or not at all, polymerizable under the temperature and pressure conditions used during step 1) of the process in accordance with the invention, that is to say which lead to a monoadduct of formula (IV) without a notable presence of diadduct, triadduct, etc. Among such monomers of formula (III), mention may in particular be made of alkenes and allylic compounds.

Among the alkenes of use as monomer of formula (III), mention may in particular be made of ethylene, propylene, 1-butene, 1-pentene, 1-hexene, 1-heptene, 1-octene, 1-nonene, 1-decene, perfluorohexylethylene and perfluorooctylethylene.

Among allylic compounds of use as monomer of formula (III), mention may in particular be made of allylic alcohol, N-allyl benzamide, ethyl N-allyl carbamate, tert-butyl N-allyl carbamate, 2-allyl-4,4,5,5-tetramethyl-1,3,2-dioxaborolane, 2-allyl-6-methyl-1,3,6,2-dioxazaborocane-4,8-dione, allylboronic acid, diethyl allylphosphonate, allyl phosphonic dichloride, dimethyl allylphosphonate, allyl cyanide, allyl isothiocyanate, allyl glycidyl ether, allyl benzyl ether, allyl phenyl ether, allyl butyl ether, allyl ethyl ether, allyl methylsulfone, allyl phenylsulfone, allyl chloride, allyl bromide and allyl fluoride.

The monomers of formula (III) may also be chosen from polymerizable compounds, in particular from styrene vinyl compounds, acrylic acid and acrylates, and acrylamide and derivatives thereof. In this case the synthesis is not selective but predominantly obtaining the monoadduct may be promoted by using a smaller amount of monomer of formula (III) than the amount of xanthate of formula (II) so as to limit as far as possible the risk of polymerization of the monomer of formula (III). Moreover, the monoadduct of formula (IV) may be isolated by conventional techniques that are within the grasp of those skilled in the art.

Among the vinyl compounds of use as monomer of formula (III), mention may in particular be made of dimethyl vinylphosphonate, diethyl vinylphosphonate, vinylphosphonic acid, N-vinyl monomers such as N-vinylpyrrolidone, N-vinylcaprolactam and N-methyl-N-vinylacetamide, vinylsulfones such as methyl vinylsulfone and phenyl vinylsulfone, vinylsilanes such as vinyltrimethylsilane and vinyltrimethoxysilane, and vinyl esters, such as vinyl acetate, vinyl pivalate, vinyl butyrate, vinyl valerate, vinyl propionate, vinyl benzoate, vinyl neodecanoate and vinyl trifluoroacetate.

Among the styrene compounds of use as monomer of formula (III), mention may in particular be made of styrene, vinylbenzoic acid, 3-chlorostyrene, 2-methylstyrene, p-t-butylstyrene, p-methoxystyrene, p-methylstyrene, p-chloromethylstyrene and vinylphenylboronic acid.

Among the acrylates, mention may in particular be made of methyl acrylate, n-butyl acrylate, t-butyl acrylate and 2-hydroxyethyl acrylate.

Among the acrylamide derivatives, mention may in particular be made of N-isopropylacrylamide, N,N-dimethylacrylamide, N-t-butylacrylamide and N-octylacrylamide.

The monomers of formula (III) are generally commercially available. When they are not commercially available, they may be readily obtained by synthesis routes that are well known to those skilled in the art.

Step 1) of the process in accordance with the invention is generally carried out at a temperature varying from approximately 10 to 140° C., and preferably from approximately 40 to 110° C., and even more preferentially between approximately 65 and 90° C.

The duration of said step 1) generally varies from approximately 3 to 48 hours, and even more preferentially from approximately 4 to 24 hours.

According to a particular and preferred embodiment form of the invention, the monoadduct of formula (IV) obtained at the end of step 1) is purified, for example by silica gel chromatography, before being used in the second step of thermolysis.

Step 2) of thermolysis of the process according to the present invention may be carried out with or without solvent. According to a preferred embodiment form of the invention, the step of thermolysis is carried out without solvent. The thermolysis temperature is generally between 40 and 210° C., preferably between 100 and 200° C. and more particularly between 160 and 190° C.

The step of thermolysis is generally carried out at a temperature sufficient to decompose the monoadduct of formula (IV).

In the present invention, the expression “thermolysis” means a thermal decomposition. This is a chemical decomposition reaction caused by heat. In the present case, the action of the heat leads to the decomposition of the monoadduct of formula (IV), enabling the formation of the thiolactone of formula (I).

In other words, step 2) of the process of the invention does not employ reagents other than the monoadduct of formula (IV) resulting from step 1). Only the action of heat makes it possible to obtain the thiolactones of formula (I).

Surprisingly, the monoadduct of formula (IV) obtained in step 1) has a suitable chemical structure, especially in terms of the definition of the groups R¹, R², R³, R⁴, R⁵, L (if m=1), Y, X, W, T (if e=1) and Z³, to enable the formation of a substituted thiolactone of formula (I) by thermolysis. In other words, cyclization to give thiolactone (I) is favoured.

When the step of thermolysis is carried out in a solvent, then said solvent is preferably chosen from high boiling point solvents (that is to say solvents having a boiling point greater than or equal to the thermolysis temperature), such as, for example, 1,2-dichlorobenzene.

Moreover, the step of thermolysis may be carried out at atmospheric pressure or under vacuum, especially in the latter case, to eliminate the volatile by-products which may have formed during the reaction.

According to a particular embodiment, the step of thermolysis is carried out in a closed container (e.g. Schlenk tube) and preferably under vacuum.

According to a particular and preferred embodiment form of the invention, the step of thermolysis is carried out without solvent and under vacuum.

At the end of step 2) of thermolysis, the thiolactone of formula (I) is preferably purified, for example by silica column chromatography.

Some of the substituted thiolactones of formula (I) that are directly obtained by carrying out the preparation process in accordance with the first subject of the invention are novel per se and thus constitute the second subject of the invention.

Therefore, another subject of the present invention is the substituted thiolactones of the following formula (I′):

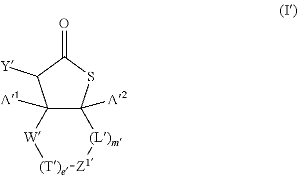

wherein:

A′¹, A′², Y′, Z^{1′}, L′, m′, T′ and e′ may respectively assume the same meanings as those defined above for A¹, A², Y, Z¹, L, m, T and e for the thiolactones of formula (I), with the proviso that:
when e′=0 and W′=H and m′=0 and Y′=hydrogen, then Z^{1′} is other than a hydrogen atom, than a linear alkyl chain or than a phenyl ring; and
when e′=0 and W′=H and m′=0 and Y′ is a substituent having a nitrogen atom directly bonded to the thiolactone ring, then Z^{1′} is other than a hydrogen atom,

in so far as such thiolactones are described in the article by Espeel P. et al., European Polymer Journal, 2015, 62, 247-272.

Among the above substituted thiolactones of formula (I′), mention may especially be made of:
dimethyl 5-oxo-tetrahydrothiophen-2-ylphosphonate (TL1);
diethyl (4-methyl-5-oxo-tetrahydrothiophen-2-yl)methylphosphonate (TL2);
diethyl (5-oxo-tetrahydrothiophen-2-yl)methylphosphonate (TL3);
3-methyl-5-pentyl-dihydrothiophen-2(3H)-one (TL4);
5-pentyl-dihydrothiophen-2(3H)-one (TL5);
3-methyl-5-(perfluorooctyl)dihydrothiophen-2(3H)-one (TL6);
3-methyl-5-phenyldihydro-2H-thieno[2,3-c]pyrrole-2,4,6(3H,5H)-trione (TL7);
3-methyl-5-(perfluorobutyl)dihydrothiophen-2(3H)-one (TL8);
(4-methyl-5-oxo-tetrahydrothiophen-2-yl)phosphonic acid (TL9);
((4-methyl-5-oxo-tetrahydrothiophen-2-yl)methyl)phosphonic acid (TL10);
(5-oxo-tetrahydrothiophen-2-yl)methylphosphonic acid (TL11);
3-methyl-5-(trimethoxysilyl)dihydrothiophen-2(3H)-one (TL12);
5-(trimethoxysilyl)dihydrothiophen-2(3H)-one (TL13);
tert-butyl-N-(4-methyl-5-oxo-tetrahydrothiophen-2-yl)carbamate (TL14);
tert-butyl (5-oxotetrahydrothiophen-2-yl)carbamate (TL15);
3-methyl-5-(oxiran-2-ylmethoxy)dihydrothiophen-2(2H)-one (TL16);
5-((oxiran-2-yloxy)methyl)dihydrothiophen-2(3H)-one (TL17);
3-methyl-5-(4,4,5,5-tetramethyl-1,3,2-dioxaborolan-2-yl)dihydrothiophen-2(3H)-one (TL18);
(5-oxo-tetrahydrothiophen-2-yl)phosphonic acid (TL19);
5-(4,4,5,5-tetramethyl-1,3,2-dioxaborolan-2-yl)dihydrothiophen-2(3H)-one (TL20);
5-(perfluorooctyl)dihydrothiophen-2(3H)-one (TL21);
5-(perfluorobutyl)dihydrothiophen-2(3H)-one (TL22);
dihydro-5-(3-(tetrahydro-4-methyl-5-oxothiophen-2-yl)propyl)-3-methylthiophen-2(3H)-one (TL23);
5-(9-hydroxynonyl)-3-methyldihydrothiophen-2(3H)-one (TL24);
5-(9-bromononyl)3-methyldihydrothiophen-2(3H)-one (TL25);
5,5′-(ethane-1,2-diyl)bis(3-methyldihydrothiophen-2(3H)-one (TL26), and
5,5′-(hexane-1,6-diyl)bis(3-methyldihydrothiophen-2(3H)-one (TL27).

The substituted thiolactones of formula (I) that are able to be obtained by carrying out the process in accordance with the present invention and in particular the thiolactones of formula (I′) in accordance with the second subject of the present invention may advantageously be used for the synthesis of polymers or for surface functionalization or polymer functionalization.

Thus, a third subject of the present invention is also the use of at least one substituted thiolactone of formula (I) obtained according to the process as defined according to the first subject of the invention, in particular of at least one substituted thiolactone of formula (I′), for the synthesis of polymers or for the functionalization of planar surfaces of metal, glass or ceramic type, particles or polymers.

Regarding the preparation of polymers, the thiolactones of formula (I) and in particular of formula (I′) may be used in a polymerization reaction comprising at least one step of reacting a thiolactone of formula (I), in particular of formula (I′), with a nucleophilic compound, making it possible to open the thiolactone ring and to obtain a thiol which may subsequently be used in an addition or condensation polymerization process with, for example, a monomer of diacrylate type such as described in the reference by Lei Yu et al, Polym. Chem., 2015, 6, 1527-1532.

Regarding the surface functionalization or polymer functionalization, it is thus possible:
according to a first embodiment form to graft substituted thiolactones of formula (I) (respectively of formula (I′)) to a solid surface or to a polymer in the liquid state, said surface or said polymer comprising chemical functions able to react with the group Z¹ or Y (respectively Z^{1′} or Y′) of the thiolactones of formula (I), respectively (I′), in order to form a covalent bond, or strong interactions of hydrogen bond type. By way of example, it is thus possible to graft a thiolactone comprising a phosphonate group, such as the phosphonic acid group of the thiolactone TL8 as group Z¹, to a metal surface. According to this first embodiment form, after functionalization, the integrity of the thiolactone ring is preserved.
according to a second embodiment form to graft the substituted thiolactone by opening the thiolactone ring on a solid surface or on a polymer in the liquid state and reacting with any substance that reacts with thiols, such as alkyl halides or acrylates for example.

Depending on the nature of the groups Y or Z¹ (respectively Y′ and Z^{1′}), it thus becomes possible to provide a material or a polymer with the properties corresponding to the type of groups Y or Z¹ (respectively Y′ or Z^{1′}) grafted, for example release properties when the groups Y or Z¹ (respectively Y′ or Z^{1′}) are perfluorinated groups. It is also possible to introduce an additional function when the thiol obtained by opening the thiolactone is reacted with a functional compound that reacts with thiols.

The present invention is illustrated by the following embodiment examples, to which, however, it is not limited.

### EXAMPLES

### Example 1: Synthesis of dimethyl 5-oxo-tetrahydrothiophen-2-ylphosphonate (TL1) According to the Process in Accordance with the Invention

In this example the thiolactone of the following formula was prepared (TL1):

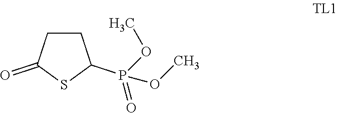

### 1) First Step: Preparation of O-1,2-dimethylpropyl S-methoxycarbonylmethyl xanthate (Xanthate of formula (II); (XA1))

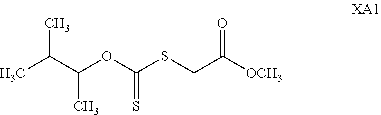

### 1.1) Sub-Step 1: Preparation of potassium O-(1,2-dimethylpropyl)xanthogenate (XA0)

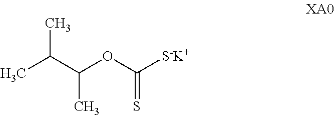

100 g (1.13 mol) of 3-methylbutan-2-ol (Alfa Aesar), 63.65 g of potassium hydroxide (KOH, Sigma-Aldrich) and carbon disulfide (CS₂, Sigma-Aldrich) were suspended in 500 ml of tetrahydrofuran (THF, Sigma-Aldrich) at room temperature for 24 hours.

After total dissolution of the KOH, the yellow emulsion was concentrated under reduced pressure then triturated with pentane (Sigma-Aldrich) and finally filtered to obtain 185 g of the expected product XA0 in the form of a yellow solid (185 g, yield 80%).

¹H NMR (300.13 MHz, D₂O, 298K) δ 5.31 (p, ³J_{H,H}=6.4 Hz, 1H, (CH₃)₂CHCH(O)CH₃); 2.07-1.84 (m, 1H, (CH₃)₂CHCH(O)CH₃); 1.27 (d, ³J_{H,H}=6.4 Hz, 3H, (CH₃)₂CHCH(O)CH₃); 0.96 (d, ³J_{H,H}=6.9 Hz, 6H, (CH₃)₂CHCH(O)CH₃).

¹³C NMR{¹H} (75.47 MHz, D₂O, 298K) δ 232.6 (C═S); 86.3 ((CH₃)₂CHCH(O)CH₃); 32.8 ((CH₃)₂CHCH(O)CH₃); 17.7 ((CH₃)₂CHCH(O)CH₃); 17.6 ((CH₃)₂CHCH(O)CH₃); 15.8 ((CH₃)₂CHCH(O)CH₃) ppm.

### 1.2) Sub-Step 2: Preparation of O-1,2-dimethylpropyl S-methoxycarbonylmethyl xanthate (XA1)

0.21 mol (32.12 g) of methyl 2-bromoacetate (Sigma-Aldrich) was added to a suspension of 40.5 g (0.2 mol) of the compound XA0 obtained above in the preceding step 1.1) in 200 ml of acetone (Sigma-Aldrich), in an ice bath. Once the addition had ended, the reaction medium was stirred at room temperature for 3 hours then filtered. The filtrate was concentrated under vacuum in order to obtain the expected product XA1 in the form of a yellow oil (42 g, yield 89%) which will be used in the following step without purification.

### 2) Second Step: Preparation of O-1,2-dimethylpropyl S-(1-(dimethylphosphoryl)-3-(methoxycarbonyl))propyl xanthate (XA1VP: Monoadduct of Formula (IV))

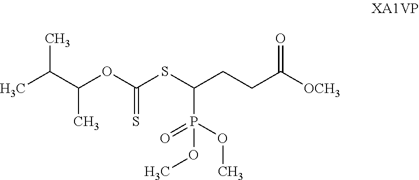 [SMILES: COC(=O)CCC(SC(=S)OC(C)C(C)C)P(=O)(OC)OC]

14.2 g (60 mmol) of the xanthate XA1 obtained above in the preceding step, 2.72 g (20 mmol) of dimethyl vinylphosphonate and 1.2 g (3 mmol) of dilauroyl peroxide (LPO: radical initiator) were mixed in a Schlenk tube. The mixture was subsequently degassed by 3 operations of freezing under vacuum. After 5 hours of heating at a temperature of 90° C., the crude reaction product was purified by silica chromatography (eluent ethyl acetate/petroleum ether (95:5, v:v) to recover the unreacted xanthate XA1, and with an eluent formed from a mixture of ethyl acetate/dichloromethane (1:4, v:v) to recover the monoadduct XA1VP (3.13 g, yield 42%, yellow oil).

¹H NMR (300.13 MHz, CDCl₃, 298K) δ 5.53-5.33 (m, 1H, (CH₃)₂CHCH(O)CH₃); 4.31-4.11 (m, 1H, CHP); 3.75-3.65 (m, 6H, O═P(OCH₃)₂); 3.59 (s, 3H, CO₂CH₃); 2.56-2.44 (m, 2H, CH₂CH₂CO₂CH₃); 2.43-1.87 (m, 2H, CH₂CH₂CO₂CH₃); 2.02-1.87 (m, 1H, (CH₃)₂CHCH(O)CH₃); 1.25-1.21 (m, 3H, (CH₃)₂CHCH(O)CH₃); 0.89-0.86 (m, 6H, (CH₃)₂CHCH(O)CH₃).

¹³C NMR{¹H} (75.47 MHz, CDCl₃, 298K) δ 211.8-211.5 (C═S); 175.7 (CO₂CH₃); 87.3; 87.2 ((CH₃)₂CHCH(O)CH₃); 53.9-53.4 (O═P(OCH₃)₂); 51.7 (CO₂CH₃); 44.6-42.5 (CHP); 32.7 ((CH₃)₂CHCH(O)CH₃); 31.0-30.8 (CH₂CH₂CO₂CH₃); 25.1-24.9 (CH₂CH₂CO₂CH₃); 18.0-17.8 ((CH₃)₂CHCH(O)CH₃); 15.7; 15.6 ((CH₃)₂CHCH(O)CH₃).

³¹P NMR{¹H} (121.49 MHz, CDCl₃, 298K) δ 26.3; 26.2 ppm.

IR: 1738.5 cm⁻¹ (C═O), 1035.1 cm⁻¹ (C═S).

Molar mass: IC(CH₄), MH⁺:

Found: 373.0916 g/mol

Calculated: 373.0908 g/mol.

### 3) Third Step: Preparation of dimethyl 5-oxo-tetrahydrothiophen-2-ylphosphonate (TL1)

3.70 g (10 mmol) of XA1VP obtained above in the preceding step were placed in a sealed Schlenk tube under vacuum and brought to a temperature of 190° C. for 15 minutes. The reaction mixture was subsequently cooled to room temperature and the volatile compounds formed were eliminated under vacuum. The thiolactone TL1 thus obtained in the form of a colourless oil (1.2 g, yield 57%) was subsequently purified on a silica chromatography column (eluent ethyl acetate/dichloromethane: 1:4 (v:v)).

¹H NMR (300.13 MHz, CDCl₃, 298K) δ 3.87-3.76 (m, 1H, CHP); 3.72-3.57 (m, 6H, O═P(OCH₃)₂); 2.63-2.33 (m, 2H, CH₂CH₂CHP); 2.32-2.17 (m, 2H, CH₂CH₂CHP).

¹³C NMR{¹H} (75.47 MHz, CDCl₃, 298K) δ 205.7-205.6 (C═O); 53.2-53.1 (O═P(OCH₃)₂); 42.3-40.2 (CHP); 39.6-39.5 (CH₂CH₂CHP); 25.40-25.35 (CH₂CH₂CHP).

³¹P NMR{¹H} (121.49 MHz, CDCl₃, 298K) δ 25.7 ppm.

IR: 1713.9 cm⁻¹ (C═O).

Molar mass: IC(CH₄), MH⁺

Found: 211.0198 g/mol

Calculated: 211.0194 g/mol.

### Example 2: Synthesis of diethyl (4-methyl-5-oxo-tetrahydrothiophen-2-yl)methylphosphonate (TL2) According to the Process in Accordance with the Invention

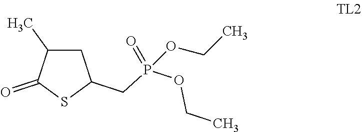 [SMILES: CCOP(=O)(CC1CC(C)C(=O)S1)OCC]

### 1) First Step: Preparation of O-1,2-dimethylpropyl S-(1-(diethylphosphoryl)-4-(methoxycarbonyl))pent-2-yl xanthate (Monoadduct of Formula (IV): XA2AP)

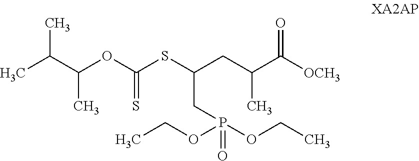 [SMILES: CCOP(=O)(CC(CC(C)C(=O)OC)SC(=S)OC(C)C(C)C)OCC]

### 1.1) Sub-Step 1: Preparation of O-1,2-dimethylpropyl S-(1-methoxycarbonyl)ethyl xanthate (XA2)

0.21 mol (35.07 g) of methyl 2-bromopropionate (Sigma-Aldrich) was added to a suspension of 40.5 g (0.2 mol) of the compound XA0 obtained above in the preceding step 1.1) of example 1, in 200 ml of acetone (Sigma-Aldrich), in an ice bath. Once the addition had ended, the reaction medium was stirred at room temperature for 3 hours then filtered. The filtrate was concentrated under vacuum in order to obtain the expected product XA2 in the form of a yellow oil (43 g, yield 86%) which will be used in the following step without purification.

¹H NMR (300.13 MHz, CDCl₃, 298K) δ 5.51 (p, ³J_{H,H}=6.3 Hz, 1H, (CH₃)₂CHCH(O)CH₃); 4.42-4.30 (m, 1H, CH(CH₃)CO₂CH₃); 3.73 (s, 3H, CO₂CH₃); 2.09-1.88 (m, 1H, (CH₃)₂CHCH(O)CH₃); 1.56-1.52 (m, ³J_{H,H}=7.4 Hz, 3H, CH(CH₃)CO₂CH₃); 1.29-1.25 (m, ³J_{H,H}=6.4 Hz, 3H, (CH₃)₂CHCH(O)CH₃); 1.02-0.91 (m, 6H, (CH₃)₂CHCH(O)CH₃).

¹³C NMR{¹H} (75.47 MHz, CDCl₃, 298K) δ 211.4; 211.4 (C═S); 172.0 (CO₂CH₃); 86.2-86.1 ((CH₃)₂CHCH(O)CH₃); 52.8 (CO₂CH₃); 46.5 (CH(CH₃)CO₂CH₃); 32.7 ((CH₃)₂CHCH(O)CH₃); 18.2-17.8 ((CH₃)₂CHCH(O)CH₃); 17.0-16.9 (CH(CH₃)CO₂CH₃); 15.8-15.7 ((CH₃)₂CHCH(O)CH₃) ppm.

IR: 1738.5 cm⁻¹ (C═O); 1046.2 cm⁻¹ (C═S)

### 1.2) Sub-Step 2: Preparation of O-1,2-dimethylpropyl S-(1-(diethylphosphoryl)-4-(methoxycarbonyl))pent-2-yl xanthate (XA2AP)

9.26 g (37 mmol) of the xanthate XA2 obtained above in step 1.1), 5.87 g (33 mmol) of diethyl allylphosphonate (DEAP, Alfa Aesar) and 2 g (5 mmol) of LPO were dissolved in 5 ml of toluene (Sigma-Aldrich). The solution thus obtained was transferred to a Schlenk tube and degassed by 3 successive operations of freezing under vacuum. After 18 hours of heating at 90° C., 10.1 g of crude reaction product were obtained in the form of a yellow oil (yield 79%) which was purified by silica chromatography (eluent ethyl acetate/dichloromethane: 1:4, v:v).

12.7 g of XA2AP were thus obtained (yield 90%).

¹H NMR (300.13 MHz, CDCl₃, 298K) δ 5.54-5.46 (m, 1H, (CH₃)₂CHCH(O)CH₃); 4.15-4.02 (m, 4H, O═P(OCH₂CH₃)₂); 4.02-3.89 (m, 1H, CHS); 3.62 (s, 3H, CO₂CH₃); 2.75-1.63 (m, 6H, CH₂P, (CH₃)₂CHCH(O)CH₃, CH₂CHCO₂CH₃); 1.32-1.23 (m, 9H, O═P(OCH₂CH₃)₂, CH₃CHCO₂CH₃); 1.17-1.14 (m, 3H, (CH₃)₂CHCH(O)CH₃); 0.92-0.90 (m, 6H, (CH₃)₂CHCH(O)CH₃).

¹³C NMR{¹H} (75.47 MHz, CDCl₃, 298K) δ 212.7-215.6 (C═S); 176.2-176.0 (CO₂CH₃); 85.8-85.7 ((CH₃)₂CHCH(O)CH₃); 62.0-61.8 (O═P(OCH₂CH₃)₂); 51.8 (CO₂CH₃); 43.8-43.6 (CHS); 37.8-36.6 (CH₂P); 37.3-37.1 ((CH₃)₂CHCH(O)CH₃); 33.4-31.1 (CH₂CH₂CO₂CH₃); 32.7 (CHCO₂CH₃); 18.3-17.9 ((CH₃)₂CHCH(O)CH₃); 18.3-17.8 (CH₃CHCO₂CH₃); 16.6-165 (O═P(OCH₂CH₃)₂); 15.9-15.8 ((CH₃)₂CHCH(O)CH₃).

³¹P NMR{¹H} (121.49 MHz, CDCl₃, 298K) δ 26.7 ppm.

IR: 1736.9 cm⁻¹ (C═O); 1043.6 cm⁻¹ (C═S)

Molar mass: IC(CH₄), MH⁺

Found: 429.1533 g/mol

Calculated: 429.1534 g/mol.

### 2) Second Step: Preparation of diethyl (4-methyl-5-oxo-tetrahydrothiophen-2-yl)methylphosphonate (TL2)

4.28 g (10 mmol) of XA2AP obtained above in the preceding step were placed in a sealed Schlenk tube under vacuum and brought to a temperature of 190° C. for 15 minutes. The reaction mixture was subsequently cooled to room temperature and the volatile compounds formed were eliminated under vacuum. The thiolactone TL2 thus obtained in the form of a colourless oil (2.4 g, yield 90%) was subsequently purified on a silica chromatography column (eluent ethyl acetate/dichloromethane: 1:4 (v:v)).

¹H NMR (300.13 MHz, CDCl₃, 298K) δ 4.18-4.05 (m, 4H, O═P(OCH₂CH₃)₂); 4.05-3.90 (m, 1H, CHCH₂P); 2.77-2.29 (m, 2H, CH₂CH(CH₃)); 2.28-2.07 (m, 2H, CHCH₂P); 1.62-1.20 (m, 1H, CHCH₂P); 1.35-1.29 (m, 6H, O═P(OCH₂CH₃)₂); 1.19-1.14 (m, 3H, CH₂CH(CH₃)).

¹³C NMR{¹H} (75.47 MHz, CDCl₃, 298K) δ 210.0-209.0 (C═O); 62.2-62.1 (O═P(OCH₂CH₃)₂); 48.7-45.4 (CHCH₂P); 42.6-42.4 (CH₂CH(CH₃)); 41.0-40.9 (CH₂CH(CH₃)); 34.2-31.7 (CHCH₂P); 16.6-16.5 (O═P(OCH₂CH₃)₂); 15.2-14.3 (CH₂CH(CH₃)).

³¹P NMR{¹H} (121.49 MHz, CDCl₃, 298K) δ 26.40, 26.36 ppm.

IR: 1700.5 cm⁻¹ (C═O); 1245.7 cm⁻¹ (P═O); 1025.4 cm⁻¹ (P—O)

Molar mass: IC(CH₄), MH⁺

Found: 267.0826 g/mol

Calculated: 267.0820 g/mol.

### Example 3: Synthesis of diethyl (5-oxo-tetrahydrothiophen-2-yl)methylphosphonate (TL3) According to the Process in Accordance with the Invention

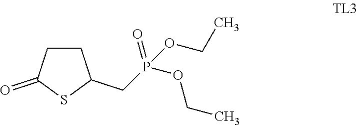

### 1) First Step: Preparation of O-1,2-dimethylpropyl S-(1-(diethylphosphoryl)-4-(methoxycarbonyl))but-2-yl xanthate (Monoadduct of Formula (IV): XA3AP)

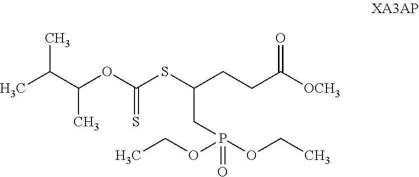 [SMILES: CCOP(=O)(CC(CCC(=O)OC)SC(=S)OC(C)C(C)C)OCC]

The monoadduct XA3AP was prepared according to the procedure used above in step 1.2) of example 2 for the preparation of the monoadduct XA2AP, but using 8.74 g (37 mmol) of the xanthate XA1 as prepared above in step 1) of example 1, 5.88 g (33 mmol) of DEAP (Sigma-Aldrich) and 2 g (5 mmol) of LPO.

12.31 g of XA3AP were thus obtained (yield 90%).

¹H NMR (300.13 MHz, CDCl₃, 298K) δ 5.49-5.39 (m, 1H, (CH₃)₂CHCH(O)CH₃); 4.09-3.96 (m, 4H, O═P(OCH₂CH₃)₂); 3.96-3.87 (m, 1H, CHS); 3.55 (s, 3H, CO₂CH₃); 2.48-1.84 (m, 7H, CH₂P, (CH₃)₂CHCH(O)CH₃, CH₂CH₂CO₂CH₃); 1.26-1.20 (m, 6H, O═P(OCH₂CH₃)₂); 1.20-1.17 (m, 3H, (CH₃)₂CHCH(O)CH₃); 0.86-0.83 (m, 6H, (CH₃)₂CHCH(O)CH₃).

¹³C NMR{¹H} (75.47 MHz, CDCl₃, 298K) δ 212.2 (C═S); 172.8 (CO₂CH₃); 85.6 ((CH₃)₂CHCH(O)CH₃); 61.9-61.7 (O═P(OCH₂CH₃)₂); 51.5 (CO₂CH₃); 44.5 (CHS); 32.5 ((CH₃)₂CHCH(O)CH₃); 32.5-30.5 (CH₂P); 31.2-31.1 (CH₂CH₂CO₂CH₃); 29.1-28.7 (CH₂CH₂CO₂CH₃); 18.1-17.8 ((CH₃)₂CHCH(O)CH₃); 16.4-16.3 (O═P(OCH₂CH₃)₂); 15.6 ((CH₃)₂CHCH(O)CH₃).

³¹P NMR{¹H} (121.49 MHz, CDCl₃, 298K) δ 26.53; 26.51 ppm.

IR: 1738.8 cm⁻¹ (C═O); 1044.9 cm⁻¹ (C═S)

Molar mass: IC(CH₄), MH⁺

Found: 415.1390 g/mol

Calculated: 415.1378 g/mol.

### 2) Second Step: Preparation of diethyl (5-oxo-tetrahydrothiophen-2-yl)methylphosphonate (TL3)

The thiolactone TL3 was prepared according to the same procedure as that used above in example 2, step 2) for the preparation of the thiolactone TL2, but using 4.14 g (10 mmol) of the monoadduct XA3AP prepared above in the preceding step instead of the monoadduct XA2AP.

2.3 g of TL3 were thus obtained in the form of a colourless oil (yield 91%).

¹H NMR (300.13 MHz, CDCl₃, 298K) δ 4.15-4.00 (m, 5H, CHCH₂P, O═P(OCH₂CH₃)₂); 2.63-2.45 (m, 3H, CH₂CH₂CO, CH₂CH₂CO); 2.29-2.08 (m, 2H, CHCH₂P); 2.05-1.89 (m, 1H, CH₂CH₂CO); 1.32-1.25 (m, 6H, O═P(OCH₂CH₃)₂).

¹³C{¹H} ¹³C NMR{¹H} (75.47 MHz, CDCl₃, 298K) δ 207.4 (C═O); 62.1-62.0 (O═P(OCH₂CH₃)₂); 44.44-44.39 (CHCH₂P); 41.8 (CH₂CH₂CO); 33.7-31.8 (CHCH₂P); 33.4-33.3 (CH₂CH₂CO); 16.5-16.4 (O═P(OCH₂CH₃)₂).

³¹P NMR{¹H} (121.49 MHz, CDCl₃, 298K) δ 26.2 ppm.

IR: 1704.4 cm⁻¹ (C═O); 1250.5 cm⁻¹ (P═O); 1025.2 cm⁻¹ (P—O)

Molar mass: IC(CH₄), MH⁺

Found: 253.0667 g/mol

Calculated: 253.0663 g/mol.

### Example 4: Synthesis of 3-methyl-5-pentyl-dihydrothiophen-2(3H)-one (TL4) According to the Process in Accordance with the Invention

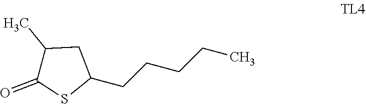

### 1) First Step: Preparation of O-1,2-dimethylpropyl S-2-(methoxycarbonyl)non-4-yl xanthate (Monoadduct of Formula (IV): XA4H)

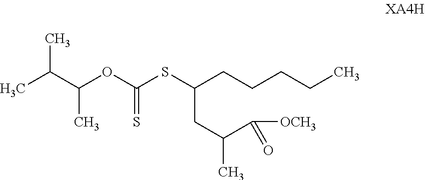 [SMILES: CCCCCC(CC(C)C(=O)OC)SC(=S)OC(C)C(C)C]

The monoadduct XA4H was prepared according to the same procedure as that used above in step 1.2) of example 2 for the preparation of the monoadduct XA2AP, but using 9.26 g (37 mmol) of the xanthate XA2 prepared in step 1.1) of example 2, 3.24 g (33 mmol) of 1-heptene (Sigma-Aldrich) and 2 g (5 mmol) of LPO.

10.7 g of XA4H were thus obtained (yellow oil; eluent ethyl acetate/petroleum ether (95:5; v:v), in the form of a racemate (yield 92%).

¹H NMR (300.13 MHz, CDCl₃, 298K) δ 5.59-5.46 (m, 1H, (CH₃)₂CHCH(O)CH₃); 3.78-3.64 (m, 1H, SCHCH₂CH(CH₃)CO₂CH₃); 3.64-3.61 (m, 3H, CO₂CH₃); 2.73-2.54 (m, 1H, SCHCH₂CH(CH₃)CO₂CH₃); 2.13-1.87 (m, 2H, SCHCH₂CH(CH₃)CO₂CH₃); 1.75-1.47 (m, 3H, (CH₃)₂CHCH(O)CH₃, CH₂(CH₂)₃CH₃); 1.47-1.31 (m, 2H, CH₂CH₂(CH₂)₂CH₃); 1.31-1.18 (m, 7H, CH(CH₃)CO₂CH₃, (CH₂)₂(CH₂)₂CH₃); 1.18-1.09 (m, 3H, (CH₃)₂CHCH(O)CH₃); 0.97-0.87 (m, 6H, (CH₃)₂CHCH(O)CH₃); 0.87-0.79 (m, 3H, (CH₂)₄CH₃).

¹³C NMR{¹H} (75.47 MHz, CDCl₃, 298K) δ 213.8-213.7 (C═S); 176.3-176.3 (CO₂CH₃); 85.2-85.0 ((CH₃)₂CHCH(O)CH₃); 51.6 (CO₂CH₃); 49.1-48.6 (SCHCH₂CH(CH₃)CO₂CH₃); 38.1-37.5 (SCHCH₂CH(CH₃)CO₂CH₃); 37.2-37.1 (SCHCH₂CH(CH₃)CO₂CH₃); 35.2-34.7 (CH₂(CH₂)₃CH₃); 32.7 ((CH₃)₂CHCH(O)CH₃); 31.6 ((CH₂)₂CH₂CH₂CH₃); 26.3-26.2 (CH₂CH₂(CH₂)₂CH₃); 22.4 ((CH₂)₃CH₂CH₃); 18.1-17.0 ((CH₃)₂CHCH(O)CH₃); 17.9-17.8 (CH(CH₃)CO₂CH₃); 15.71-15.69 ((CH₃)₂CHCH(O)CH₃); 13.9 (SCH(CH₂)₄CH₃) ppm.

IR: 1738.5 cm⁻¹ (C═O); 1047.2 cm⁻¹ (C═S).

Molar mass: IC(CH₄), MH⁺

Found: 349.1861 g/mol

Calculated: 349.1871 g/mol.

### 2) Second Step: Preparation of 3-methyl-5-pentyl-dihydrothiophen-2(3H)-one (TL4)

The thiolactone TL4 was prepared according to the same procedure as that used above in example 2, step 2) for the preparation of the thiolactone TL2, but using 3.48 g (10 mmol) of the monoadduct XA4H prepared above in the preceding step instead of the monoadduct XA2AP.

1.73 g of thiolactone TL4 were thus obtained (yield 93%) in the form of a colourless liquid (eluent: ethyl acetate/petroleum ether: 9:1; v:v).

¹H NMR (300 MHz, CDCl₃, 298K) δ 3.79-3.64 (m, 1H, SCHCH₂CH); 2.76-2.46 (m, 2H, SCHCH₂CH); 2.20-1.97 (m, 1H, SCHCH₂CH); 1.83-1.62 (m, 2H, CH₂CH₂(CH₂)₂CH₃); 1.55-1.35 (m, 2H, CH₂(CH₂)₃CH₃); 1.35-1.21 (m, 4H, (H₂)₂(CH₂)₂CH₃); 1.20-1.13 (m, 3H, CHCH₃); 0.92-0.84 (m, 3H, (CH₂)₄CH₃).

¹³C NMR{¹H} (75.47 MHz, CDCl₃, 298K) δ 211.2-210.0 (C═O); 48.92-47.89 (SCHCH₂CH); 47.5-456 (SCHCH₂CH); 415-39.7 (SCHCH₂CH); 36.8-36.5 (CH₂(CH₂)₃CH₃); 31.7-31.6 ((CH₂)₂CH₂CH₂CH₃); 28.2-28.0 (CH₂CH₂(CH₂)₂CH₃); 22.60-22.56 ((CH₂)₃CH₂CH₃); 15.5-14.6 (CHCH₃); 14.1 ((CH₂)₄CH₃) ppm.

IR: 1700.9 cm⁻¹ (C═O).

Molar mass: IE

Found: 186.1075 g/mol

Calculated: 186.1078 g/mol.

### Example 5: Synthesis of 5-pentyl-dihydrothiophen-2(3H)-one (TL5) According to the Process in Accordance with the Invention

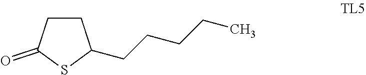

### 1) First Step: Preparation of O-1,2-dimethylpropyl S-1-(methoxycarbonyl)oct-3-yl xanthate (Monoadduct of Formula (IV): XA5H)

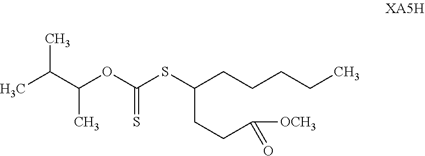 [SMILES: CCCCCC(CCC(=O)OC)SC(=S)OC(C)C(C)C]

The monoadduct XA5H was prepared according to the procedure used above in step 1.2) of example 2 for the preparation of the monoadduct XA2AP, but using 8.74 g (37 mmol) of the xanthate XA1 as prepared above in step 1.2) of example 1, 3.24 g (33 mmol) of 1-heptene and 2 g (5 mmol) of LPO.

10 g of thiolactone XA5H were thus obtained in the form of a yellow oil (eluent: ethyl acetate/petroleum ether: 95:5; v:v) in the form of a racemate (yield 90%).

¹H NMR (300.13 MHz, CDCl₃, 298K) δ 5.53 (p, ³J_{H,H}=6.3 Hz, 1H, (CH₃)₂CHCH(O)CH₃); 3.78-3.66 (m, 1H, SCHCH₂CH₂CO₂CH₃); 3.64 (s, 3H, CO₂CH₃); 2.49-2.36 (m, 2H, SCHCH₂CH₂CO₂CH₃); 2.12-1.94 (m, 2H, CH₂(CH₂)₃CH₃); 1.93-1.77 (m, 1H, (CH₃)₂CHCH(O)CH₃); 1.69-1.52 (m, 2H, SCHCH₂CH₂CO₂CH₃); 1.52-1.33 (m, 2H, CH₂CH₂(CH₂)₂CH₃); 1.33-1.16 (m, 7H, (CH₂)₂(CH₂)₂CH₃; (CH₃)₂CHCH(O)CH₃); 0.93 (d, J=6.8 Hz, 6H, (CH₃)₂CHCH(O)CH₃); 0.89-0.79 (m, 3H, (CH₂)₄CH₃).

¹³C NMR{¹H} (75.47 MHz, CDCl₃, 298K) δ 214.0-213.9 (C═S); 173.5-173.5 (CO₂CH₃); 85.4-85.4 ((CH₃)₂CHCH(O)CH₃); 51.7 (CO₂CH₃); 50.3-50.2 (SCHCH₂CH₂CO₂CH₃); 34.6-34.3 (CH₂(CH₂)₃CH₃); 32.8 ((CH₃)₂CHCH(O)CH₃); 31.7 ((CH₂)₂CH₂CH₂CH₃); 31.5-31.4 (SCHCH₂CH₂CO₂CH₃); 29.7-29.3 (SCHCH₂CH₂CO₂CH₃); 26.6-26.5 (CH₂CH₂(CH₂)₂CH₃); 22.6 ((CH₂)₃CH₂CH₃); 18.3-18.0 ((CH₃)₂CHCH(O)CH₃); 15.8 ((CH₃)₂CHCH(O)CH₃); 14.1 ((CH₂)₄CH₃) ppm.

IR: 1740.9 cm⁻¹ (C═O); 1048.6 cm⁻¹ (C═S).

Molar mass: IC(CH₄), MH⁺

Found: 335.1701 g/mol

Calculated: 335.1715 g/mol

### 2) Second Step: Preparation of 5-pentyl-dihydrothiophen-2(3H)-one (TL5)

The thiolactone TL5 was prepared according to the same procedure as that used above in example 2, step 2) for the preparation of the thiolactone TL2, but using 3.34 g (10 mmol) of the monoadduct XA5H prepared above in the preceding step instead of the monoadduct XA2AP.

1.55 g of TL5 were thus obtained in the form of a colourless liquid (eluent: ethyl acetate/petroleum ether: 9:1; v:v) (yield 90%).

¹H NMR (300.13 MHz, CDCl₃, 298K) δ 3.82 (tt, ³J_{H,H}=8.5; 5.8 Hz, 1H, SCHCH₂CH₂); 2.66-2.44 (m, 2H, SCHCH₂CH₂); 2.43-1.79 (m, 2H, SCHCH₂CH₂); 1.79-1.62 (m, 2H, CH₂CH₂(CH₂)₂CH₃); 1.47-1.33 (m, 2H, CH₂(CH₂)₃CH₃); 1.32-1.19 (m, 4H, (CH₂)₂(CH₂)₂CH₃); 0.86 (t, ³J_{H,H}=7.0 Hz, 3H, (CH₂)₄CH₃).

¹³C NMR{¹H} (75.47 MHz, CDCl₃, 298K) δ 208.6 (C═O); 51.4 (SCHCH₂CH₂); 42.1 (SCHCH₂CH₂); 36.5 (CH₂(CH₂)₃CH₃); 32.3 ((CH₂)₂CH₂CH₂CH₃); 31.5 (SCHCH₂CH₂); 28.0 (CH₂CH₂(CH₂)₂CH₃); 22.5 ((CH₂)₃CH₂CH₃); 14.0 ((CH₂)₄CH₃) ppm.

IR: 1704.6 cm⁻¹ (C═O)

Mass: IE

Found: 172.0910 g/mol

Calculated: 172.0922 g/mol.

### Example 6: Synthesis of 3-methyl-5-(perfluorooctyl)dihydrothiophen-2(3H)-one (TL6) According to the Process in Accordance with the Invention

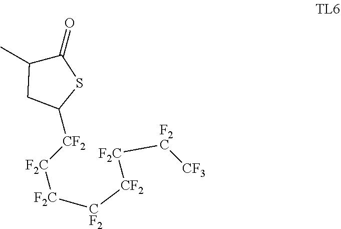 [SMILES: CC1CC(C(F)(F)C(F)(F)C(F)(F)C(F)(F)C(F)(F)C(F)(F)C(F)(F)C(F)(F)F)SC1=O]

### 1) First Step: Preparation of Methyl 5,5,6,6,7,7,8,8,9,9,10,10,11,11,12,12,12-heptadecafiuoro-2-methyl-4-((((3-methylbutan-2-yl)oxy)carbonothioyl)thio)dodecanoate (Monoadduct of Formula (IV): XA6DF)

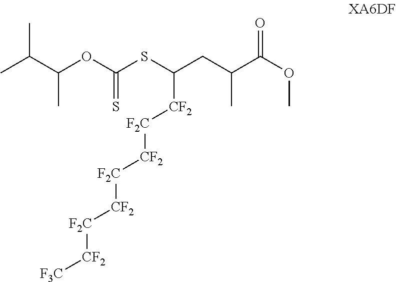 [SMILES: COC(=O)C(C)CC(SC(=S)OC(C)C(C)C)C(F)(F)C(F)(F)C(F)(F)C(F)(F)C(F)(F)C(F)(F)C(F)(F)C(F)(F)F]

The monoadduct XA6DF was prepared according to the procedure used above in step 1.2) of example 2 for the preparation of the monoadduct XA2AP, but using 3.09 g (1.23×10⁻² mol) of xanthate XA2 as prepared above in step 1.1) of example 2, 5 g (1.12×10⁻² mol) of 1H,1H,2H-perfluoro-1-decene (Aldrich), 0.67 g of LPO, and a reaction solvent composed of 4 ml of toluene and 3 ml of trifluorotoluene.

The monoadduct XA6DF obtained in the form of a viscous yellow oil was purified by silica chromatography (eluent hexane/ethyl acetate: 9:1, v:v).

3.2 g of XA6DF were thus obtained (yield 41%).

¹H NMR (300.13 MHz, CDCl₃, 298K): δ (ppm) 5.58-5.47 (s, 1H, (CH₃)₂CHCH(O)CH₃); 4.87-4.67 (m, 1H, SCHCH₂CH(CH₃)); 3.70-3.68 (m, 3H, CO₂CH₃); 2.92-2.68 (1H, m, SCHCH₂CH(CH₃)); 2.14-1.92 (m, 1H, (CH₃)₂CHCH(O)CH₃); 1.58-1.53 (m, 2H, SCHCH₂CH(CH₃)); 1.32-1.20 (m, 6H; (CH₃)₂CHCH(O)CH₃ et SCHCH₂CH(CH₃)); 0.97-0.93 (m, 6H, (CH₃)₂CHCH(O)CH₃).

¹³C NMR{¹H} (75.47 MHz, CDCl₃, 298K): δ (ppm) 209.33 (C═S); 175.54 (CO₂CH₃); 129.01-115.52 (CH(CF₂)₇CF₃); 87.48 ((CH₃)₂CHCH(O)CH₃); 52.71-51.79 (CO₂CH₃); 46.50 (CH(CF₂)₃CF₃); 36.48 (SCHCH₂CH(CH₃)); 32.71 ((CH₃)₂CHCH(O)CH₃); 18.30-15.44 ((CH₃)₂CHCH(O)CH₃ et (SCHCH₂CH(CH₃)).

NMR ¹⁹F{¹H} (282.38 MHz, CDCl₃, 298K): δ (ppm) −80.83 (CH(CF₂—CF₂—CF₂—CF₂—CF₂—CF₂—CF₂—CF₃)); −105.65-−116.25 (CH(CF₂—CF₂—CF₂—CF₂—CF₂—CF₂—CF₂—CF₃)); −118.87-−119.33 (CH(CF₂—CF₂—CF₂—CF₂—CF₂—CF₂—CF₂—CF₃)); −121.69-−121.91 (CH(CF₂—CF₂—CF₂—CF₂—CF₂—CF₂—CF₂—CF₃)); −122.72 (CH(CF₂—CF₂—CF₂—CF₂—CF₂—CF₂—CF₂—CF₃)); −126.16 (CH(CF₂—CF₂—CF₂—CF₂—CF₂—CF₂—CF₂—CF₃)).

### 2) Second Step: Preparation of 3-Methyl-5-(perfluorooctyl)dihydrothiophen-2(3H)-one (TL6)

2 g (2.87 mmol) of XA6DF obtained above in the preceding step were placed in a sealed Schlenk tube under vacuum and brought to a temperature of 190° C. for 48 hours. The reaction mixture was subsequently cooled to room temperature and the volatile compounds formed were eliminated under vacuum. The thiolactone TL6 thus obtained in the form of a yellow oil was subsequently purified on a silica chromatography column (eluent ethyl acetate/hexane: 1:1 (v:v)).

0.8 g of TL6 were thus obtained in the form of a white powder (yield 52%).

¹H NMR (300.13 MHz, CDCl₃, 298K): δ (ppm) 4.49-4.36 (m, 1H, CHCF₂); 2.75-2.59 (m, 2H, C(O)CH(CH₃)CH₂CH); 1.99-1.87 (q, 1H, C(O)CH(CH₃)CH₂CH); 1.26-1.23 (d, 3H, C(O)CH(CH₃)).

NMR ¹⁹F{¹H} (282.38 MHz, CDCl₃, 298K): δ (ppm) −81.03 (CH(CF₂—CF₂—CF₂—CF₂—CF₂—CF₂—CF₂—CF₃)); −112.13-−119.71 (CH(CF₂—CF₂—CF₂—CF₂—CF₂—CF₂—CF₂—CF₃)); −121.08 (CH(CF₂—CF₂—CF₂—CF₂—CF₂—CF₂—CF₂—CF₃)); −121.96 (CH(CF₂—CF₂—CF₂—CF₂—CF₂—CF₂—CF₂—CF₃)); −122.86 (CH(CF₂—CF₂—CF₂—CF₂—CF₂—CF₂—CF₂—CF₃)); −126.29 (CH(CF₂—CF₂—CF₂—CF₂—CF₂—CF₂—CF₂—CF₃)).

### Example 7: Synthesis of 3-methyl-5-phenyldihydro-2H-thieno[2,3-c]pyrrole-2,4,6(3H,5H)-trione (TL7) According to the Process in Accordance with the Invention

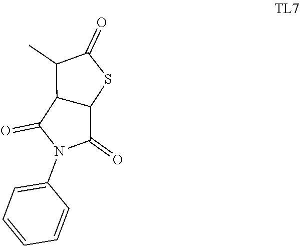 [SMILES: CC1C(=O)SC2C(=O)N(c3ccccc3)C(=O)C12]

### 1) First Step: Preparation of XA7MAL (Monoadduct of Formula (IV))

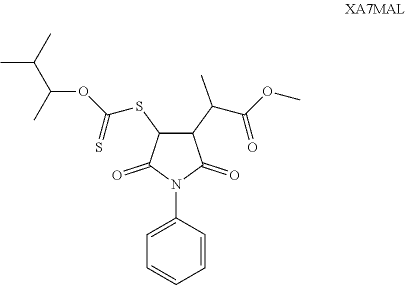 [SMILES: COC(=O)C(C)C1C(=O)N(c2ccccc2)C(=O)C1SC(=S)OC(C)C(C)C]

The monoadduct XA7MAL was prepared according to the procedure used above in step 1.2) of example 2 for the preparation of the monoadduct XA2AP, but using 4.77 g (1.91×10⁻² mol) of the xanthate XA2 as prepared above in step 1.1) of example 2, 3 g (1.73×10⁻² mol) of N-phenylmaleimide (Aldrich) and 1.03 g of LPO.

The monoadduct XA7MAL obtained in the form of a viscous yellow oil was purified by silica chromatography (eluent hexane/ethyl acetate: 7:3, v:v).

4.9 g of XA7MAL were thus obtained (yield 68%).

¹H NMR (300.13 MHz, CDCl₃, 298K): δ (ppm) 7.47-7.30 (m, 5H, N—C₆H₅); 5.57-5.48 (s, 1H, (CH₃)₂CHCH(O)CH₃); 5.22-4.07 (m, 1H, SCH(CO)CH); 3.76-3.66 (m, 3H, CO₂CH₃); 3.55-3.21 (m, 2H, SCH(CO)CH(CO)CH(CH₃)); 2.07-1.92 (1H, (CH₃)₂CHCH(O)CH₃); 1.56-1.39 (m, 3H, CH(CO)CH(CH₃)); 1.39-1.26 (m, 3H, (CH₃)₂CHCH(O)CH₃); 0.98-0.92 (m, 3H, (CH₃)₂CHCH(O)CH₃).

¹³C NMR{¹H} (75.47 MHz, CDCl₃, 298K): δ (ppm) 209.77 (C═S); 175.59-171.71 (C(O)NC(O) et CO₂CH₃); 131.38-126.36 (N—C₆H₅); 87.27 ((CH₃)₂CHCH(O)CH₃); 52.42 CO₂CH₃); 50.47-46.87 (SCH(CO)CH(CO)CH(CH₃)); 39.71-38.99 (SCH(CO)CH(CO)CH(CH₃)); 32.82 ((CH₃)₂CHCH(O)CH₃); 18.34-17.84 ((CH₃)₂CHCH(O)CH₃); 15.81-15.21 (SCH(CO)CH(CO)CH(CH₃)).

### 2) Second Step: Preparation of 3-methyl-5-phenyldihydro-2H-thieno[2,3-c]pyrrole-2,4,6(3H,5H)-trione (TL7)

1 g (2.36 mmol) of XA7MAL obtained above in the preceding step was placed in a sealed Schlenk tube under vacuum and brought to a temperature of 190° C. for 48 hours. The reaction mixture was subsequently cooled to room temperature and the volatile compounds formed were eliminated under vacuum. The thiolactone TL7 thus obtained in the form of a yellow oil was subsequently purified on a silica chromatography column (eluent ethyl acetate/hexane: 1:1 (v:v)).

0.4 g of TL7 were thus obtained (yield 65%).

¹H NMR (300.13 MHz, CDCl₃, 298K): δ (ppm) 7.55-7.30 (m, 5H, N—C₆H₅); 4.86-4.75 (s, 1H, SCH(CO)CH); 3.46-3.44 (m, 1H, CH(CH₃)CHC(O)); 3.34-3.25 (m, 1H, C(O)CH(CH₃)); 1.49-1.47 (d, 3H, C(O)CH(CH₃)).

¹³C NMR{¹H} (75.47 MHz, CDCl₃, 298K): δ (ppm) 205.25 (SC═O); 174.5-173.49 (C(O)NC(O)); 129.21-126.39 (N—C₆H₅) 49.54 (SCH(CO)); 49.03 (C(O)CH(CH₃)); 46.81 (C(O)CH(CH₃)CH(CO); 18.41 (C(O)CH(CH₃)CH₂).

### Example 8: Synthesis of 3-methyl-5-(perfluorobutyl)dihydrothiophen-2(3H)-one (TL8) According to the Process in Accordance with the Invention

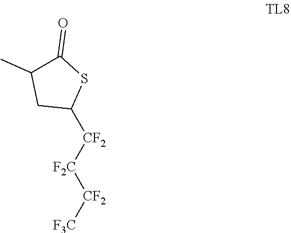 [SMILES: CC1CC(C(F)(F)C(F)(F)C(F)(F)C(F)(F)F)SC1=O]

### 1) First Step: Preparation of methyl 5,5,6,6,7,7,8,8,8-nonafluoro-2-methyl-4-((((3-méthylbutan-2-yl)oxy)carbonothioyl)thio)octanoate (Monoadduct of Formula (IV): XA8HF)

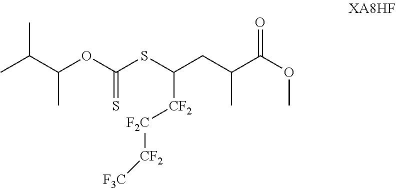 [SMILES: COC(=O)C(C)CC(SC(=S)OC(C)C(C)C)C(F)(F)C(F)(F)C(F)(F)C(F)(F)F]

The monoadduct XA8HF was prepared according to the procedure used above in step 1.2) of example 2 for the preparation of the monoadduct XA2AP, but using 4.47 g (1.78×10⁻² mol) of the xanthate XA2 as prepared above in step 1.1) of example 2, 4 g (1.62×10⁻² mol) of 1H,1H,2H-perfluoro-1-hexene (Aldrich) and 0.969 g of LPO.

The monoadduct XA8HF obtained in the form of a viscous yellow oil was purified by silica chromatography (eluent hexane/ethyl acetate: 9:1, v:v).

3.73 g of XA8HF were thus obtained (yield 46%).

¹H NMR (300.13 MHz, CDCl₃, 298K): δ (ppm) 5.59-5.48 (s, 1H, (CH₃)₂CHCH(O)CH₃); 4.92-4.66 (m, 1H, SCHCH₂CH(CH₃)); 3.73-3.67 (m, 3H, CO₂CH₃); 2.91-2.61 (1H, m, SCHCH₂CH(CH₃)); 2.16-1.88 (m, 1H, (CH₃)₂CHCH(O)CH₃); 1.60-1.52 (m, 2H, SCHCH₂CH(CH₃)); 1.32-1.20 (m, 6H, (CH₃)₂CHCH(O)CH₃ et SCHCH₂CH(CH₃)); 0.96-0.92 (m, 6H, (CH₃)₂CHCH(O)CH₃).

¹³C NMR{¹H} (75.47 MHz, CDCl₃, 298K): δ (ppm) 209.30 (C═S); 175.35 (CO₂CH₃); 129.01-127.51 (CH(CF₂)₃CF₃); 87.49 ((CH₃)₂CHCH(O)CH₃); 52.70-51.91 (CO₂CH₃); 46.48 CH(CF₂)₃CF₃); 36.46 (SCHCH₂CH(CH₃)), 32.64 ((CH₃)₂CHCH(O)CH₃); 18.32-14.08 ((CH₃)₂CHCH(O)CH₃ et (SCHCH₂CH(CH₃)).

NMR ¹⁹F{¹H} (282.38 MHz, CDCl₃, 298K): δ (ppm) −80.80 (CH(CF₂—CF₂—CF₂—CF₃)), −105.96-−116.42 (CH(CF₂—CF₂—CF₂—CF₃)), −119.86-−120.36 (CH(CF₂—CF₂—CF₂—CF₃)), −125.60-−126.30 (CH(CF₂—CF₂—CF₂—CF₃)).

### 2) Second Step: Preparation of 3-methyl-5-(perfluorobutyl)dihydrothiophen-2(3H)-one (TL8)

2 g (4.03 mmol) of XA8HF obtained above in the preceding step were placed in a sealed Schlenk tube under vacuum and brought to a temperature of 190° C. for 48 hours. The reaction mixture was subsequently cooled to room temperature and the volatile compounds formed were eliminated under vacuum. The thiolactone TL8 thus obtained in the form of a colourless oil was subsequently purified on a silica chromatography column (eluent ethyl acetate/hexane: 5:95 (v:v)).

0.8 g of TL8 were thus obtained in the form of a white powder (yield 52%).

¹H NMR (300.13 MHz, CDCl₃, 298K): δ (ppm) 4.49-4.36 (m, 1H, CHCF₂); 2.75-2.59 (m, 2H, C(O)CH(CH₃)CH₂CH); 1.99-1.87 (q, 1H, C(O)CH(CH₃)CH₂CH); 1.26-1.23 (d, 3H, C(O)CH(CH₃)).

NMR ¹⁹F{¹H} (282.38 MHz, CDCl₃, 298K): δ (ppm) −81.03 (CH(CF₂—CF₂—CF₂—CF₂—CF₂—CF₂—CF₂—CF₃)); −112.13-−119.71 (CH(CF₂—CF₂—CF₂—CF₂—CF₂—CF₂—CF₂—CF₃)); −121.08 (CH(CF₂—CF₂—CF₂—CF₂—CF₂—CF₂—CF₂—CF₃)); −121.96 (CH(CF₂—CF₂—CF₂—CF₂—CF₂—CF₂—CF₂—CF₃)); −122.86 (CH(CF₂—CF₂—CF₂—CF₂—CF₂—CF₂—CF₂—CF₃)); −126.29 (CH(CF₂—CF₂—CF₂—CF₂—CF₂—CF₂—CF₂—CF₃)).

### Example 9: Functionalization of Polymers by a Thiolactone According to the Invention

### 1) Example 9.1: Functionalization of bis(3-aminopropyl)-poly(dimethylsiloxane) by TL4

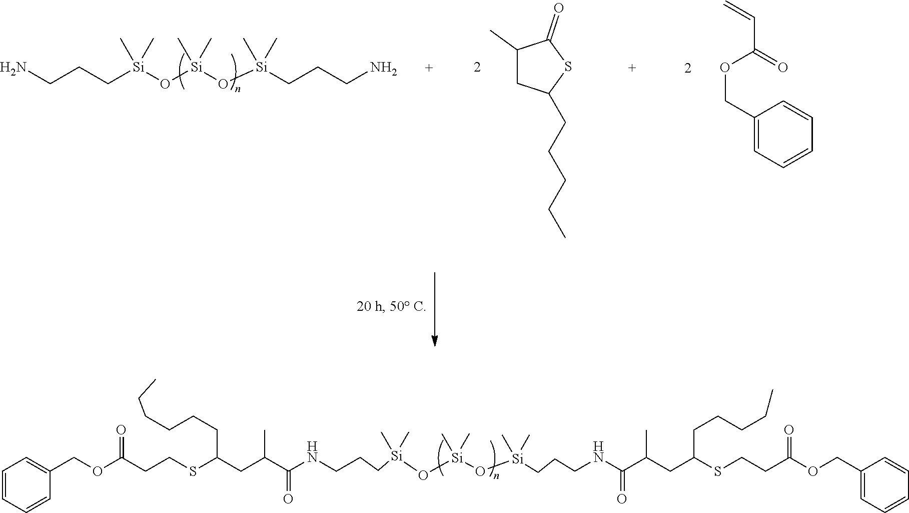

The following were successively introduced into a 25 ml round-bottomed flask: bis(3-aminopropyl)-poly(dimethylsiloxane) (2500 g·mol⁻¹, 500 mg, 2×10⁻⁴ mol, Aldrich), TL4 (52.1 mg, 2.8×10⁻⁴ mol) and benzyl acrylate (45.4 mg, 2.8×10⁻⁴ mol, Aldrich). The reaction mixture was stirred for 20 hours at 50° C. ¹H NMR analysis and MALDI-TOF analysis showed total functionalization of the bis(3-aminopropyl)-poly(dimethylsiloxane).

### 2) Example 9.2: Functionalization of Methoxypolyethylene Glycol Amine by TL4

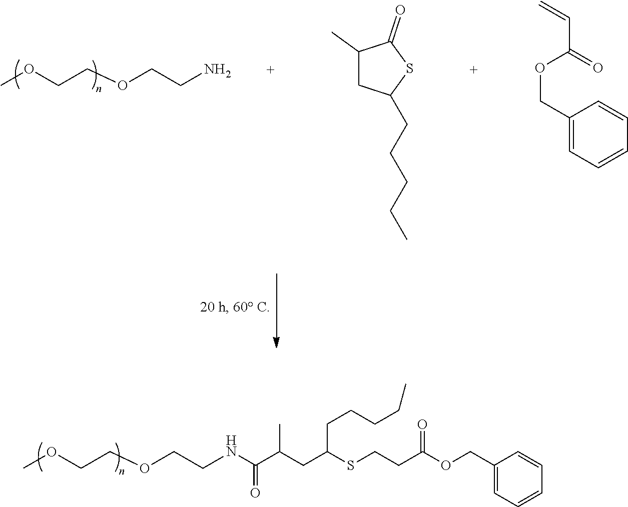

The following were successively introduced into a 25 ml round-bottomed flask: methoxypolyethylene glycol amine (2000 g·mol⁻¹, 200 mg, 1×10⁻⁴ mol, Aldrich), TL4 (16.7 mg, 9×10⁻⁵ mol) and benzyl acrylate (14.6 mg, 9×10⁻⁵ mol, Aldrich). The reaction mixture was stirred for 20 hours at 60° C. ¹H NMR analysis and MALDI-TOF analysis showed total functionalization of the methoxypolyethylene glycol amine.

### 3) Example 9.3: Functionalization of bis(3-aminopropyl)-poly(dimethylsiloxane) by TL2

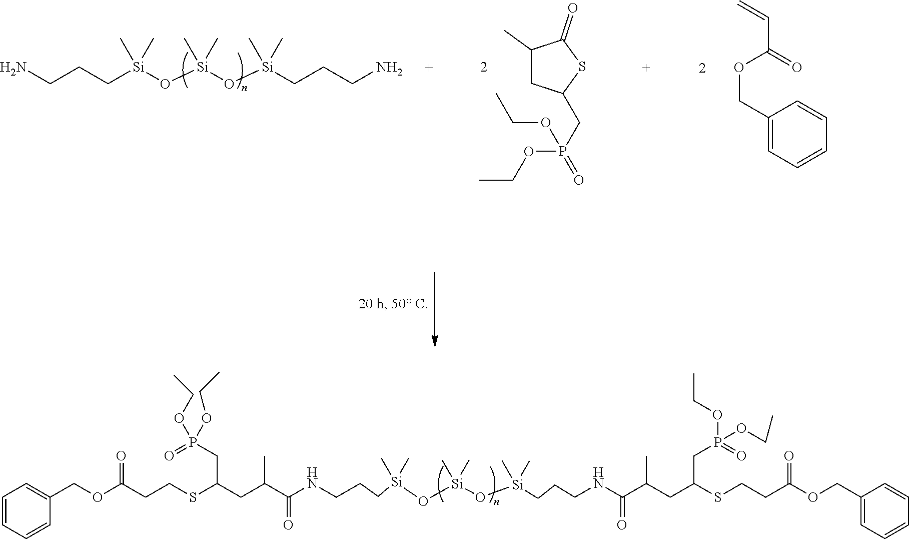

The following were successively introduced into a 25 ml round-bottomed flask: bis(3-aminopropyl)-poly(dimethylsiloxane) (2500 g·mol⁻¹, 500 mg, 2×10⁻⁴ mol, Aldrich), TL2 (74.5 mg, 2.8×10⁴ mol) and benzyl acrylate (45.4 mg, 2.8×10⁻⁴ mol, Aldrich). The reaction mixture was stirred for 20 hours at 50° C. ¹H NMR analysis showed total functionalization of the bis(3-aminopropyl)-poly(dimethylsiloxane).

### 4) Example 9.4: Functionalization of methoxypolyethylene glycol amine by TL2

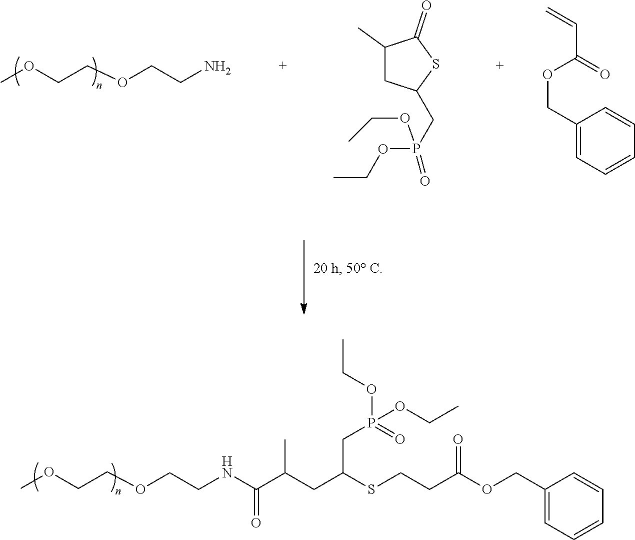

The following were successively introduced into a 25 ml round-bottomed flask: methoxypolyethylene glycol amine (2000 g·mol⁻¹, 200 mg, 1×10⁻⁴ mol, Aldrich), TL2 (23.9 mg, 9×10⁻⁵ mol) and benzyl acrylate (14.6 mg, 9×10⁻⁵ mol, Aldrich). The reaction mixture was stirred for 20 hours at 60° C. ¹H NMR analysis showed total functionalization of the methoxypolyethylene glycol amine.

### 5) Example 9.5: Polymerization of bis(3-aminopropyl)-poly(dimethylsiloxane) with TL2 and poly(ethylene glycol) diacrylate

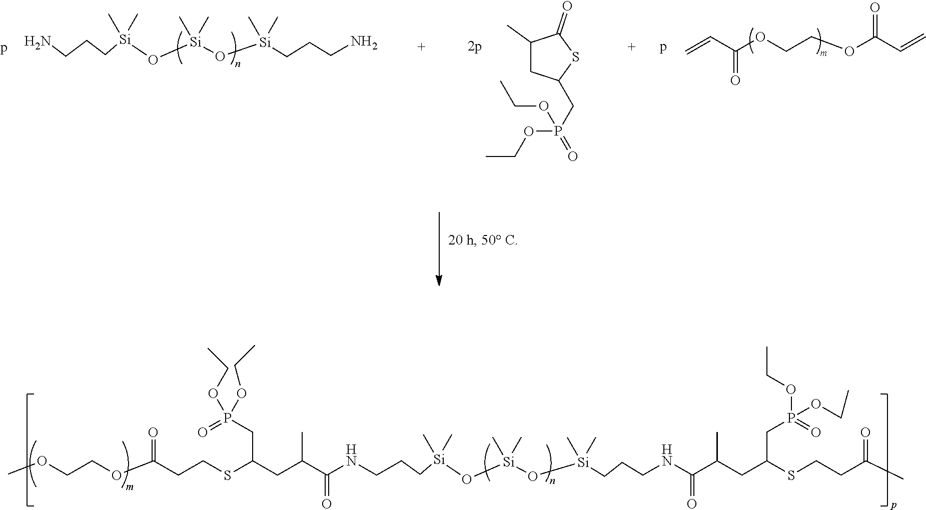

The following were successively introduced into a 25 ml round-bottomed flask: bis(3-aminopropyl)-poly(dimethylsiloxane) (2500 g·mol⁻¹, 500 mg, 2.0×10−4 mol, Aldrich), TL2 (74.5 mg, 2.8×10⁻⁴ mol) and poly(ethylene glycol) diacrylate (575 g·mol⁻¹, 80.5 mg, 1.4×10−4 mol, Aldrich). The reaction mixture was stirred for 20 hours at 50° C. H and ³¹P NMR analysis showed total consumption of the monomers, and size exclusion chromatography confirmed the formation of the polymer. Mn_{(PS)}=8100 g·mol⁻¹ Mw_{(PS)}=14 500 g·mol⁻¹.

### Example 10: Synthesis of dihydro-5-(3-(tetrahydro-4-methyl-5-oxothiophen-2-yl)propyl)-3-methylthiophen-2(3H)-one (TL23) According to the Process in Accordance with the Invention

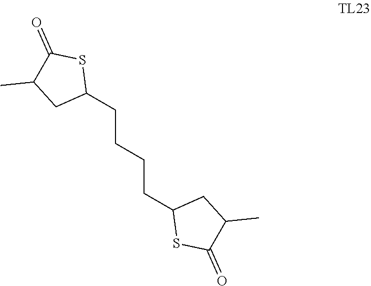

### 1) First Step: Preparation of the Monoadduct of Formula (IVa); XA23OD

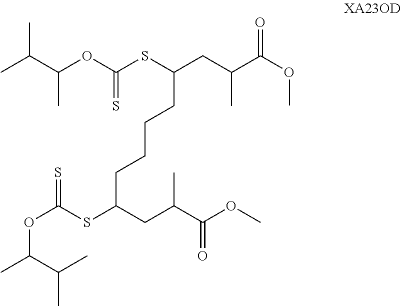 [SMILES: COC(=O)C(C)CC(CCCCC(CC(C)C(=O)OC)SC(=S)OC(C)C(C)C)SC(=S)OC(C)C(C)C]

The monoadduct XA23OD was prepared according to the procedure used above in step 1.2) of example 2 for the preparation of the monoadduct XA2AP, but using 4.77 g (1.90×10⁻² mol) of xanthate XA2 as prepared in step 1.2) of example 2, 1 g (0.90×10⁻² mol) of 1,7-octadiene (Aldrich), 0.54 g of LPO, and a reaction solvent composed of 2 ml of toluene.

The monoadduct XA23OD obtained in the form of a viscous yellow oil was purified by silica chromatography (eluent hexane/ethyl acetate: 95:5, v:v).

2.49 g of XA23OD were thus obtained (yield 45%).

¹H NMR (300.13 MHz, CDCl₃, 298K): δ (ppm) 5.47-5.46 (s, 2H, (CH₃)₂CHCH(O)CH₃); 3.77-3.78 (m, 2H, SCHCH₂CH(CH₃)); 3.66-3.64 (m, 6H, CO₂CH₃); 2.73-2.61 (2H, m, SCHCH₂CH(CH₃)); 2.15-1.91 (m, 4H, SCHCH₂CH(CH₃)); 1.73-1.49 (m, 6H, (CH₃)₂CHCH(O)CH₃ et SCH(CH₂CH₂)CH₂CH(CH₃)); 1.49-1.34 (m, 4H, SCH(CH₂CH₂)CH₂CH(CH₃)); 1.28-1.23 (d, 3H, SCHCH₂CH(CH₃)); 1.17-1.15 (d, 3H, (CH₃)₂CHCH(O)CH₃); 0.94-0.92 (m, 6H, (CH₃)₂CHCH(O)CH₃).

### 2) Second Step: Preparation of dihydro-5-(3-(tetrahydro-4-methyl-5-oxothiophen-2-yl)propyl)-3-methylthiophen-2(3H)-one (TL23)

2.49 g (4.1 mmol) of XA23OD obtained above in the preceding step were placed in a sealed Schlenk tube under vacuum and brought to a temperature of 190° C. for 48 hours. The reaction mixture was subsequently cooled to room temperature and the volatile compounds formed were eliminated under vacuum. The thiolactone TL9 thus obtained in the form of a yellow oil was subsequently purified on a silica chromatography column (eluent ethyl acetate/hexane: 8:2 (v:v)).

0.877 g of TL23 were thus obtained in the form of a white powder (yield 75%).

¹H NMR (300.13 MHz, CDCl₃, 298K): δ (ppm) 3.78-3.62 (m, 2H, CHS); 2.75-2.43 (m, 2H, C(O)CH(CH₃)CH₂CH); 2.20-1.99 (m, 2H, C(O)CH(CH₃)CH₂CH); 1.84-1.63 (m, 4H, CH₂CH₂CH₂); 1.56-1.38 (m, 5H, CHCH₂CH₂, CHCH₂CH₂); 1.17-1.13 (m, 6H, CH₃).

¹³C NMR{¹H} (282.38 MHz, CDCl₃, 298K): δ (ppm) 210.74-209.61 (C═O); 48.78-45.42 ((CH₃)CHCH₂CH₂CHS); 41.30-39.48 ((CH₃)CHCH₂CH₂CHS); 36.59-36.13 (CHCH₂CH₂CH₂); 28.26-27.94 (CHCH₂CH₂CH₂); 15.38-14.45 (CH₃).

### Example 11: Synthesis of 5-(9-hydroxynonyl)-3-methyldihydrothiophen-2(3H)-one (TL 24) According to the Process in Accordance with the Invention

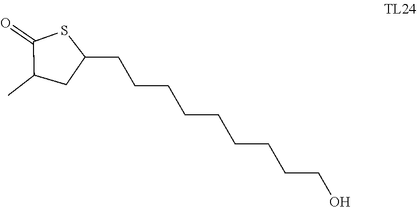 [SMILES: CC1CC(CCCCCCCCCO)SC1=O]

### 1) First Step: Preparation of methyl 13-hydroxy-2-methyl-4-((((3-methylbutan-2-yl)oxy)carbonothioyl)thio)tridecanoate (Xanthate XA2HN)

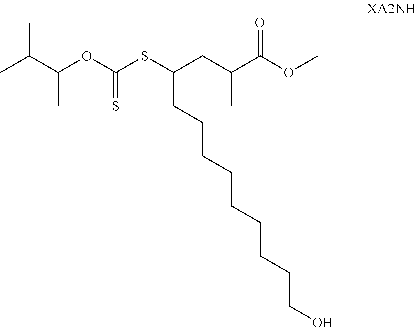 [SMILES: COC(=O)C(C)CC(CCCCCCCCCO)SC(=S)OC(C)C(C)C]

### 1.1) Sub-Step 1: Preparation of O-1,2-dimethylpropyl S-(1-methoxycarbonyl)ethyl xanthate (XA2)

0.21 mol (35.07 g) of methyl 2-bromopropionate (Sigma-Aldrich) was added to a suspension of 40.5 g (0.2 mol) of the compound XA0 obtained above in the preceding step 1.1) of example 1, in 200 ml of acetone (Sigma-Aldrich), in an ice bath (highly exothermic reaction). Once the addition had ended, the reaction medium was stirred at room temperature for 3 hours then filtered. The filtrate was concentrated under vacuum in order to obtain the expected product XA2 in the form of a yellow oil (43 g, yield 86%) which will be used in the following step without purification.

¹H NMR (300.13 MHz, CDCl₃, 298K) δ 5.51 (p, ³J_{H,H}=6.3 Hz, 1H, (CH₃)₂CHCH(O)CH₃); 4.42-4.30 (m, 1H, CH(CH₃)CO₂CH₃); 3.73 (s, 3H, CO₂CH₃); 2.09-1.88 (m, 1H, (CH₃)₂CHCH(O)CH₃); 1.56-1.52 (m, ³J_{H,H}=7.4 Hz, 3H, CH(CH₃)CO₂CH₃); 1.29-1.25 (m, ³J_{H,H}=6.4 Hz, 3H, (CH₃)₂CHCH(O)CH₃); 1.02-0.91 (m, 6H, (CH₃)₂CHCH(O)CH₃).

¹³C NMR{¹H} (75.47 MHz, CDCl₃, 298K) δ 211.4; 211.4 (C═S); 172.0 (CO₂CH₃); 86.2-86.1 ((CH₃)₂CHCH(O)CH₃); 52.8 (CO₂CH₃); 46.5 (CH(CH₃)CO₂CH₃); 32.7 ((CH₃)₂CHCH(O)CH₃); 18.2-17.8 ((CH₃)₂CHCH(O)CH₃); 17.0-16.9 (CH(CH₃)CO₂CH₃); 15.8-15.7 ((CH₃)₂CHCH(O)CH₃) ppm.

IR: 1738.5 cm⁻¹ (C═O); 1046.2 cm⁻¹ (C═S)

### 1.2) Sub-step 2: Preparation of methyl 13-hydroxy-2-methyl-4-((((3-methylbutan-2-yl)oxy)carbonothioyl)thio)tridecanoate (XA2HN)

The xanthate XA2HN was prepared according to the same procedure as that used above in step 1.2) of example 2 for the preparation of the xanthate XA2AP, but using 5.01 g (20 mmol) of the xanthate XA2 prepared in the preceding step, 3.05 g (18 mmol) of undecene-1-ol (Sigma-Aldrich) and 1 g (26 mmol) of LPO.

5.8 g of XA2HN were thus obtained (yellow oil; eluent hexane/ethyl acetate (7:3; v:v), in the form of a racemate (yield 77%).

¹H NMR (300.13 MHz, CDCl₃, 298K) δ=5.57-5.48 (m, 1H, (CH₃)₂CHCH(O)CH₃); 3.75-3.71 (m, 1H, SCHCH₂CH(CH₃)CO₂CH₃); 3.66-3.64 (m, 3H, CO₂CH₃); 3.62,-3.57 (t, 2H, CH₂(CH₂)₆CH₂CH₂OH); 2.72-2.65 (m, 1H, SCHCH₂CH(CH₃)CO₂CH₃); 2.12-1.91 (m, 2H, SCHCH₂CH(CH₃)CO₂CH₃); 1.80 (s, 1H, OH); 1.73-1.48 (m, 5H, (CH₃)₂CHCH(O)CH₃; CH₂(CH₂)₆CH₂CH₂OH); 1.38-1.24 (m, 15H, CH(CH₃)CO₂CH₃; CH₂(CH₂)₆CH₂CH₂OH); 1.17-1.15 (m, 3H, (CH₃)₂CHCH(O)CH₃); 0.94-0.92 (m, 6H, (CH₃)₂CHCH(O)CH₃).

¹³C NMR{¹H} (75.47 MHz, CDCl₃, 298K) δ=213.80-213.70 (C═S); 176.65 (CO₂CH₃); 85.40 ((CH₃)₂CHCH(O)CH₃); 62.90 (CH₂(CH₂)₆CH₂CH₂OH); 51.58 (CO₂CH₃); 49.14-48.59 (SCHCH₂CH(CH₃)CO₂CH₃); 38.16-37.54 (SCHCH₂CH(CH₃)CO₂CH₃); 37.50-37.13 (SCHCH₂CH(CH₃)CO₂CH₃); 32.70-32.69 ((CH₃)₂CHCH(O)CH₃; CH₂(CH₂)₆CH₂CH₂OH); 29.48-25.72 (CH₂(CH₂)₆CH₂CH₂OH); 18.25-18.23 ((CH₃)₂CHCH(O)CH₃); 17.02-15.79 ((CH₃)₂CHCH(O)CH₃); CH(CH₃)CO₂CH₃).

### 2) Second Step: Preparation of 5-(9-hydroxynonyl)-3-methyldihydrothiophen-2(3H)-one (TL24)

The thiolactone TL24 was prepared according to the same procedure as that used above in example 2, step 3) for the preparation of the thiolactone TL2, but using 3.4 g (8 mmol) of the xanthate XA2HN prepared above in the preceding step instead of the xanthate XA2AP.

1.89 g of thiolactone TL24 were thus obtained (yield 91%) in the form of a colourless liquid (eluent: hexane/ethyl acetate: 5:5; v:v).

¹H NMR (300 MHz, CDCl₃, 298K) δ=3.74-3.63 (m, 1H, SCHCH₂CH); 3.58-3.53 (t, 2H, SCHCH₂(CH₂)₆CH₂CH₂OH); 2.72-2.43 (m, 1.5H, C(O)CH(CH₃)CH₂); 2.24 (s, 1H, OH); 2.15-1.95 (m, 1H, SCHCH₂CH); 1.74-1.60 (m, 2H, CH₂(CH₂)₆CH₂CH₂OH); 1.52-1.42 (m, 2.5H, SCHCH₂CH, CH₂(CH₂)₆CH₂CH₂OH); 1.37-1.20 (m, 12H, CH₂(CH₂)₆CH₂CH₂OH); 1.12-1.09 (m, 3H, C(O)CH(CH₃)CH₂).

¹³C NMR{¹H} (75.47 MHz, CDCl₃, 298K) δ=211.34-210.18 (C═O); 62.78 (CH₂(CH₂)₆CH₂CH₂OH); 48.81-45.50 (SCHCH₂CH, C(O)CH(CH₃)CH₂); 41.37-39.52 (SCHCH₂(CH₂)₆CH₂CH₂OH); 36.71-36.36 (C(O)CH(CH₃)CH₂); 32.70 (SCHCH₂(CH₂)₆CH₂CH₂OH); 29.45-25.73 (SCHCH₂(CH₂)₆CH₂CH₂OH); 15.38-14.42 (C(O)CH(CH₃)CH₂).

### Example 12: Synthesis of 5-(9-bromononyl)-3-methyldihydrothiophen-2(3H)-one (TL25) According to the Process in Accordance with the Invention

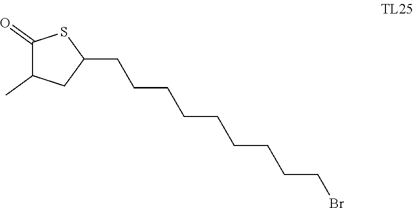 [SMILES: CC1CC(CCCCCCCCCBr)SC1=O]

### 1) First Step: Preparation of methyl 13-bromo-2-methyl-4-((((3-methylbutan-2-yl)oxy)carbonothioyl)thio)tridecanoate (Xanthate XA2BN)

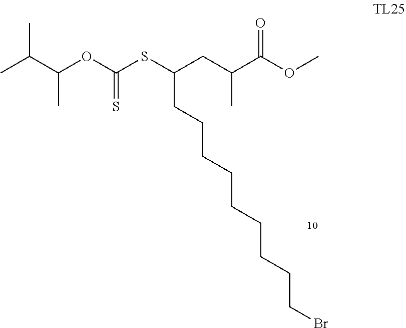 [SMILES: COC(=O)C(C)CC(CCCCCCCCCBr)SC(=S)OC(C)C(C)C]

### 1.1) Sub-Step 1: Preparation of O-1,2-dimethylpropyl S-(1-methoxycarbonyl)ethyl xanthate (XA2)

0.21 mol (35.07 g) of methyl 2-bromopropionate (Sigma-Aldrich) was added to a suspension of 40.5 g (0.2 mol) of the compound XA0 obtained above in the preceding step 1.1) of example 1, in 200 ml of acetone (Sigma-Aldrich), in an ice bath (highly exothermic reaction). Once the addition had ended, the reaction medium was stirred at room temperature for 3 hours then filtered. The filtrate was concentrated under vacuum in order to obtain the expected product XA2 in the form of a yellow oil (43 g, yield 86%) which will be used in the following step without purification.

¹H NMR (300.13 MHz, CDCl₃, 298K) δ 5.51 (p, ³J_{H,H}=6.3 Hz, 1H, (CH₃)₂CHCH(O)CH₃); 4.42-4.30 (m, 1H, CH(CH₃)CO₂CH₃); 3.73 (s, 3H, CO₂CH₃); 2.09-1.88 (m, 1H, (CH₃)₂CHCH(O)CH₃); 1.56-1.52 (m, ³J_{H,H}=7.4 Hz, 3H, CH(CH₃)CO₂CH₃); 1.29-1.25 (m, ³J_{H,H}=6.4 Hz, 3H, (CH₃)₂CHCH(O)CH₃); 1.02-0.91 (m, 6H, (CH₃)₂CHCH(O)CH₃).

¹³C NMR{¹H} (75.47 MHz, CDCl₃, 298K) δ 211.4; 211.4 (C═S); 172.0 (CO₂CH₃); 86.2-86.1 ((CH₃)₂CHCH(O)CH₃); 52.8 (CO₂CH₃); 46.5 (CH(CH₃)CO₂CH₃); 32.7 ((CH₃)₂CHCH(O)CH₃); 18.2-17.8 ((CH₃)₂CHCH(O)CH₃); 17.0-16.9 (CH(CH₃)CO₂CH₃); 15.8-15.7 ((CH₃)₂CHCH(O)CH₃) ppm.

IR: 1738.5 cm⁻¹ (C═O); 1046.2 cm⁻¹ (C═S)

### 1.2) Sub-Step 2: Preparation of methyl 13-bromo-2-methyl-4-((((3-methylbutan-2-yl)oxy)carbonothioyl)thio)tridecanoate (XA2BN)

The xanthate XA2BN was prepared according to the same procedure as that used above in step 1.2) of example 2 for the preparation of the xanthate XA2AP, but using 1.01 g (4 mmol) of the xanthate XA2 prepared in the preceding step, 0.86 g (3.7 mmol) of bromo-11-undecene (Alfa-Aesar) and 0.22 g (0.5 mmol) of LPO.

1.41 g of XA2BN were thus obtained (yellow oil; eluent hexane/ethyl acetate (95:5; v:v), in the form of a racemate (yield 80%).

¹H NMR (300.13 MHz, CDCl₃, 298K) δ=5.59-5.52 (m, 1H, (CH₃)₂CHCH(O)CH₃); 3.78-3.77 (m, 1H, SCHCH₂CH(CH₃)CO₂CH₃); 3.66-3.64 (m, 3H, CO₂CH₃); 3.41-3.36 (t, 2H, CH₂(CH₂)₆CH₂CH₂Br); 2.70-2.63 (m, 1H, SCHCH₂CH(CH₃)CO₂CH₃); 2.08-1.91 (m, 2H, SCHCH₂CH(CH₃)CO₂CH₃); 1.73-1.48 (m, 5H, (CH₃)₂CHCH(O)CH₃; CH₂(CH₂)₆CH₂CH₂Br); 1.38-1.24 (m, 15H, CH(CH₃)CO₂CH₃, CH₂(CH₂)₆CH₂CH₂Br); 1.17-1.15 (m, 3H, (CH₃)₂CHCH(O)CH₃); 0.94-0.92 (m, 6H, (CH₃)₂CHCH(O)CH₃).

¹³C NMR{¹H} (75.47 MHz, CDCl₃, 298K) δ=213.80-213.70 (C═S); 176.65 (CO₂CH₃); 85.40 ((CH₃)₂CHCH(O)CH₃); 51.58 (CO₂CH₃); 49.14-48.59 (SCHCH₂CH(CH₃)CO₂CH₃); 38.16-37.54 (SCHCH₂CH(CH₃)CO₂CH₃); 37.50-37.13 (SCHCH₂CH(CH₃)CO₂CH₃); 34.90 (CH₂(CH₂)₆CH₂CH₂Br); 32.70-32.69 ((CH₃)₂CHCH(O)CH₃; CH₂(CH₂)₆CH₂CH₂Br); 29.48-25.72 (CH₂(CH₂)₆CH₂CH₂Br); 18.25-18.23 ((CH₃)₂CHCH(O)CH₃); 17.02-15.79 ((CH₃)₂CHCH(O)CH₃); CH(CH₃)CO₂CH₃).

### 2) Second Step: Preparation of 5-(9-bromononyl)-3-methyldihydrothiophen-2(3H)-one (TL25)

The thiolactone TL25 was prepared according to the same procedure as that used above in example 2, step 3) for the preparation of the thiolactone TL2, but using 0.59 g (1.2 mmol) of the xanthate XA2BN prepared above in the preceding step instead of the xanthate XA2AP.

0.202 g of thiolactone TL25 were thus obtained (yield 52%) in the form of a colourless liquid (eluent: hexane/ethyl acetate: 9:1; v:v).

¹H NMR (300 MHz, CDCl₃, 298K) δ=3.79-3.66 (m, 1H, SCHCH₂CH); 3.42-3.38 (t, 2H, SCHCH₂(CH₂)₆CH₂CH₂Br); 2.74-2.49 (m, 1.5H, C(O)CH(CH₃)CH₂); 2.18-2.02 (m, 1H, SCHCH₂CH); 1.89-1.59 (m, 4H, CH₂(CH₂)₆CH₂CH₂Br); 1.37-1.20 (m, 13H, CH₂(CH₂)₆CH₂CH₂Br); 1.12-1.09 (m, 3H, C(O)CH(CH₃)CH₂).

¹³C NMR{¹H} (75.47 MHz, CDCl₃, 298K) δ=211.05-209.91 (C═O); 48.82-45.0 (SCHCH₂CH, C(O)CH(CH₃)CH₂); 41.40 (CH₂(CH₂)₆CH₂CH₂Br); 39.56-39.52 (SCHCH₂(CH₂)₆CH₂CH₂Br); 36.76-36.40 (C(O)CH(CH₃)CH₂); 32.70 (SCHCH₂(CH₂)₆CH₂CH₂Br); 29.45-25.73 (SCHCH₂(CH₂)₆CH₂CH₂Br); 15.38-14.42 (C(O)CH(CH₃)CH₂).

### Example 13: Synthesis of 5,5′-(ethane-1,2-diyl)bis(3-methyldihydrothiophen-2(3H)-one) (TL 26) According to the Process in Accordance with the Invention

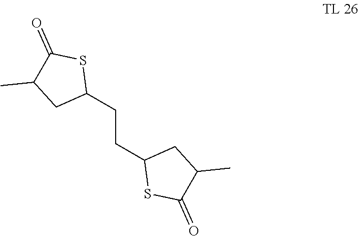

### 1) First Step: Preparation of the Monoadduct of Formula (IVa); XA2ED

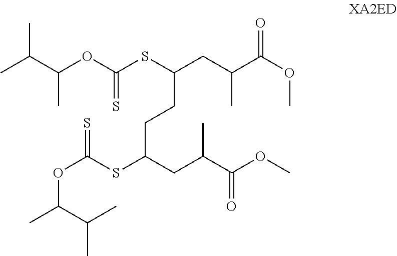 [SMILES: COC(=O)C(C)CC(CCC(CC(C)C(=O)OC)SC(=S)OC(C)C(C)C)SC(=S)OC(C)C(C)C]

The monoadduct XA2ED was prepared according to the procedure used above in step 1.2) of example 2 for the preparation of the monoadduct XA2AP, but using 8.01 g (3.2×10⁻² mol) of xanthate XA2 as prepared in step 1.2) of example 2, 1.25 g (1.5×10⁻² mol) of 1,5-hexadiene (Aldrich), 1.77 g of LPO, and a reaction solvent composed of 6 ml of toluene.

The monoadduct XA2ED obtained in the form of a viscous yellow oil was purified by silica chromatography (eluent hexane/ethyl acetate: 95:5, v:v).

3.79 g of XA2ED were thus obtained (yield 73%).

¹H NMR (300.13 MHz, CDCl₃, 298K): δ (ppm) 5.47-5.46 (s, 2H, (CH₃)₂CHCH(O)CH₃); 3.77-3.78 (m, 2H, SCHCH₂CH(CH₃)); 3.66-3.64 (m, 6H, CO₂CH₃); 2.73-2.61 (2H, m, SCHCH₂CH(CH₃)); 2.15-1.91 (m, 4H, SCHCH₂CH(CH₃)); 1.73-1.49 (m, 6H, (CH₃)₂CHCH(O)CH₃ et SCH(CH₂CH₂)CH₂CH(CH₃)); 1.28-1.23 (d, 3H, SCHCH₂CH(CH₃)); 1.17-1.15 (d, 3H, (CH₃)₂CHCH(O)CH₃); 0.94-0.92 (m, 6H, (CH₃)₂CHCH(O)CH₃).

### 2) Second Step: Preparation of 5,5′-(ethane-1,2-diyl)bis(3-methyldihydrothiophen-2(3H)-one) (TL26)

3.36 g (5.7 mmol) of XA2ED obtained above in the preceding step were placed in a sealed Schlenk tube under vacuum and brought to a temperature of 190° C. for 48 hours. The reaction mixture was subsequently cooled to room temperature and the volatile compounds formed were eliminated under vacuum. The thiolactone TL26 thus obtained in the form of a yellow oil was subsequently purified on a silica chromatography column (eluent ethyl acetate/hexane: 6:4 (v:v)).

1.2 g of TL26 were thus obtained in the form of a white powder (yield 84%).

¹H NMR (300.13 MHz, CDCl₃, 298K): δ (ppm) 3.78-3.62 (m, 2H, CHS); 2.75-2.43 (m, 2H, C(O)CH(CH₃)CH₂CH); 2.20-1.99 (m, 2H, C(O)CH(CH₃)CH₂CH); 1.56-1.38 (m, 5H, CHCH₂CH₂, CHCH₂CH₂); 1.17-1.13 (m, 6H, CH₃).

¹³C NMR{¹H} (282.38 MHz, CDCl₃, 298K): δ (ppm) 210.74-209.61 (C═O); 48.78-45.42 ((CH₃)CHCH₂CH₂CHS); 41.30-39.48 ((CH₃)CHCH₂CH₂CHS); 36.59-36.13 (CHCH₂CH₂CH₂); 15.38-14.45 (CH₃).

### Example 14: Synthesis of 5,5′-(hexane-1,6-diyl)bis(3-methyldihydrothiophen-2(3H)-one) (TL 27) According to the Process in Accordance with the Invention

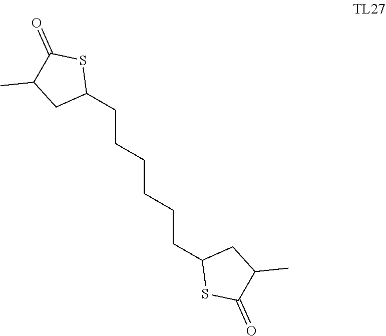

### 1) First Step: Preparation of a Monoadduct of Formula (IVa); XA2HD

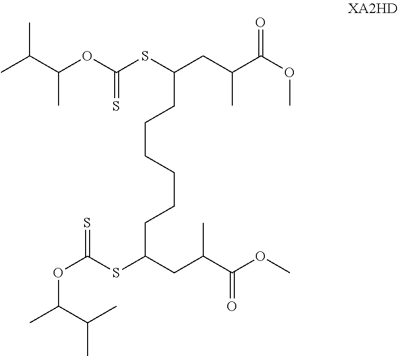 [SMILES: COC(=O)C(C)CC(CCCCCCC(CC(C)C(=O)OC)SC(=S)OC(C)C(C)C)SC(=S)OC(C)C(C)C]

The monoadduct XA2HD was prepared according to the procedure used above in step 1.2) of example 2 for the preparation of the monoadduct XA2AP, but using 9.07 g (3.6×10⁻² mol) of xanthate XA2 as prepared in step 1.2) of example 2, 2.38 g (1.5×10⁻² mol) of 1,9-decadiene (Aldrich), 2.03 g of LPO, and a reaction solvent composed of 7 ml of toluene.

The monoadduct XA2HD obtained in the form of a viscous yellow oil was purified by silica chromatography (eluent hexane/ethyl acetate: 90:10, v:v). 7.37 g of XA2HD were thus obtained (yield 68%).

¹H NMR (300.13 MHz, CDCl₃, 298K): δ (ppm) 5.58-5.49 (s, 2H, (CH₃)₂CHCH(O)CH₃); 3.77-3.78 (m, 2H, SCHCH₂CH(CH₃)); 3.66-3.64 (m, 6H, CO₂CH₃); 2.73-2.61 (2H, m, SCHCH₂CH(CH₃)); 2.15-1.91 (m, 4H, SCHCH₂CH(CH₃)); 1.73-1.28 (m, 14H, (CH₃)₂CHCH(O)CH₃ et SCH(CH₂CH₂CH₂CH₂CH₂CH₂)CHSH) et (CH₃)₂CHCH(O)CH₃); 1.28-1.23 (d, 3H, SCHCH₂CH(CH₃)); 1.17-1.15 (d, 3H, (CH₃)₂CHCH(O)CH₃); 0.94-0.92 (m, 6H, (CH₃)₂CHCH(O)CH₃).

### 2) Second Step: Preparation of 5,5′-(hexane-1,6-diyl)bis(3-methyldihydrothiophen-2(3H)-one (TL27)

6.56 g (10.2 mmol) of XA2HD obtained above in the preceding step were placed in a sealed Schlenk tube under vacuum and brought to a temperature of 190° C. for 48 hours. The reaction mixture was subsequently cooled to room temperature and the volatile compounds formed were eliminated under vacuum. The thiolactone TL27 thus obtained in the form of a yellow oil was subsequently purified on a silica chromatography column (eluent ethyl acetate/hexane: 7:3 (v:v)).

2.48 g of TL27 were thus obtained in the form of a white powder (yield 77%).

¹H NMR (300.13 MHz, CDCl₃, 298K): δ (ppm) 3.78-3.62 (m, 2H, CHS); 2.75-2.43 (m, 2H, C(O)CH(CH₃)CH₂CH); 2.20-1.99 (m, 2H, C(O)CH(CH₃)CH₂CH); 1.78-1.64 (m, 4H, SCH(CH₂CH₂CH₂CH₂CH₂CH₂)CHSH) 1.53-1.38 (m, 9H, CHCH₂CH₂, SCH(CH₂CH₂CH₂CH₂CH₂CH₂)CHSH); 1.17-1.13 (m, 6H, CH₃).

¹³C NMR{¹H} (282.38 MHz, CDCl₃, 298K): δ (ppm) 210.74-209.61 (C═O); 48.78-45.42 ((CH₃)CHCH₂CHS); 41.30-39.48 ((CH₃)CHCH₂CHS); 36.71-36.36 (SCH(CH₂CH₂CH₂CH₂CH₂CH₂)CHSH); 28.34-28.16 (SCH(CH₂CH₂CH₂CH₂CH₂CH₂)CHCHSH); 15.38-14.45 (CH₃).

1. Process for preparing substituted thiolactones of the following formula (I): 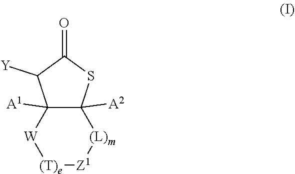
wherein:
A¹ and A², which are identical or different, represent a hydrogen atom or a fluorine atom,
Y represents a hydrogen atom or a group selected from alkyl, hydroxyalkyl, aryl and cyano groups, or a polymer chain;
L is a linker arm,
m is an integer equal to 0 or 1,
T represents CH₂, —O— or —NR⁶—, in which R⁶ represents a hydrogen atom or an alkyl, aryl or aralkyl radical, optionally substituted by a group selected from the groups: maleimide, a group of formula: 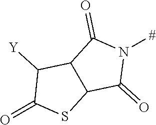
in which the symbol # is the point of attachment of said group to R⁶ and in which Y has the same meaning as that chosen for the radical Y of the formula (I), OH; P(O)(OR⁷)(OR^{7′}) in which the radicals R⁷ and R^{7′}, which are identical or different, represent a hydrogen atom or an alkyl radical; CₙF₂ₙ₊₁ in which n is an integer ranging from 1 to 20; SiR⁸ₚ(OR⁹)₃₋ₚ, in which the radicals R⁸ and R⁹, which are identical or different, represent a hydrogen atom or an alkyl radical and p is an integer equal to 0, 1 or 2; BF₃M⁺, in which M=K or Na; B(OR¹⁰)₂, in which the two radicals R¹⁰, which are identical or different, represent a hydrogen atom, an alkyl radical or form a carbon-based ring with the two oxygen atoms to which they are bonded; OR¹¹, in which R¹¹ represents a hydrogen atom or an alkyl, aryl or aralkyl radical; O(C═O)R¹², in which R¹² represents a hydrogen atom or an alkyl, aryl or aralkyl radical; O(C═O)OR¹³, in which R¹³ represents a hydrogen atom or an alkyl, aryl or aralkyl radical; N⁺R¹⁴R^{14′}R^{14″}A⁻, in which the radicals R¹⁴, R^{14′} and R^{14″}, which are identical or different, represent a hydrogen atom or an alkyl, aryl or aralkyl radical and A represents a chlorine or bromine atom; NR^{15′}(C═O)R¹⁵, in which the radicals R¹⁵ and R^{15′}, which are identical or different, represent a hydrogen atom or an alkyl or aryl radical or are connected together and form a ring such as a pyrrolidone or caprolactam ring; NR^{16′}(C═O)OR¹⁶, in which R¹⁶ et R^{16′}, which are identical or different, represent a hydrogen atom or an alkyl, aryl or aralkyl radical; CN; a halogen atom chosen from Cl, F, and Br; NCS; OCH₂-epoxy; COOR¹⁷, in which R¹⁷ represents a hydrogen atom, an alkyl, aryl or aralkyl radical; CONR¹⁸R^{18′}, in which R¹⁸ and R^{18′}, which are identical or different, represent a hydrogen atom or an alkyl or aryl radical; SO₂R¹⁹, in which R¹⁹ represents an alkyl or aryl radical; azide N₃ and alkyne,
e is an integer equal to 0 or 1,
wherein:
1) when A¹=A²=H and e=0, then W represents a hydrogen atom and Z¹ represents a group selected from the groups alkyl; aryl; P(O)(OR⁷)(OR^{7′}), in which the radicals R⁷ and R^{7′}, which are identical or different, represent a hydrogen atom or an alkyl radical; CₙF₂ₙ₊₁ in which n is an integer ranging from 1 to 20; SiR⁸ₚ(OR⁹)₃₋ₚ, in which the radicals R⁸ and R⁹, which are identical or different, represent a hydrogen atom or an alkyl radical and p is an integer equal to 0, 1 or 2; BF₃M⁺, in which M=K or Na; B(OR¹⁰)₂, in which the two radicals R¹⁰, which are identical or different, represent a hydrogen atom, an alkyl radical or form a carbon-based ring with the two oxygen atoms to which they are bonded; OR¹¹, in which R¹¹ represents a hydrogen atom or an alkyl, aryl or aralkyl radical; O(C═O)R¹², in which R¹² represents a hydrogen atom or an alkyl, aryl or aralkyl radical; O(C═O)OR¹³, in which R¹³ represents a hydrogen atom or an alkyl, aryl or aralkyl radical; N⁺R¹⁴R^{14′}R^{14″}A⁻, in which the radicals R¹⁴, R^{14′} and R^{14″}, which are identical or different, represent a hydrogen atom or an alkyl, aryl or aralkyl radical and A represents a chlorine or bromine atom; NR^{15′}(C═O)R¹⁵, in which the radicals R¹⁵ and R^{15′}, which are identical or different, represent a hydrogen atom or an alkyl or aryl radical or are connected together and form a ring such as a pyrrolidone or caprolactam ring; NR^{16′}(C═O)OR¹⁶, in which R¹⁶ and R^{16′}, which are identical or different, represent a hydrogen atom or an alkyl, aryl or aralkyl radical; CN; a halogen atom chosen from Cl, F, and Br; NCS; OCH₂-epoxy; COOR¹⁷, in which R¹⁷ represents a hydrogen atom, an alkyl, aryl or aralkyl radical; CONR¹⁸R^{18′}, in which R¹⁸ and R^{18′}, which are identical or different, represent a hydrogen atom or an alkyl or aryl radical; SO₂R¹⁹, in which R¹⁹ represents an alkyl or aryl radical; azide N₃ and alkyne; and a thiolactone ring of formula 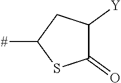
in which the symbol # is the point of attachment of the thiolactone ring to L and in which Y has the same meaning as that chosen for the radical Y of the formula (I),
2) when A¹=A²=H, m=1 and e=0, then W represents a hydrogen atom and Z¹ may also represent a hydrogen atom;
3) when A¹=A²=H, e=1 and m=0, then Z¹ and W are identical and represent —CH₂— or CO;
4) when A¹=A²=H, e=1, m=1 and T=CH₂, then Z¹=W=—CH₂,
5) when A¹=A²=F, e=0 and m=0, then W represents a fluorine atom and Z¹=F or represents a linear or branched chain OC_{q}F_{2q+1} in which q is an integer ranging from 1 to 5, [OCF₂CF(CF₃)]ᵣOC₃F₇, with r=an integer ranging from 0 to 20, OC₂F₄SO₂F, OCF₂CF(CF₃)OC₂F₄SO₂F, or OCF₂CF(CF₃)OC₂F₄CO₂CH₃; and
6) when A¹=A²=F, e=1 and m=0, then W=Z¹=CF₂ and T=(CF₂)ₛ, with s=an integer ranging from 1 to 5;
7) when A¹=H, A²=F, and e=m=0, then W represents a hydrogen atom and Z¹=F or CₙF₂ₙ₊₁, in which n is an integer ranging from 1 to 20;
wherein said process comprises at least the following steps:
1) a step during which, in the presence of a radical initiator, a xanthate of the following formula (II) is reacted: 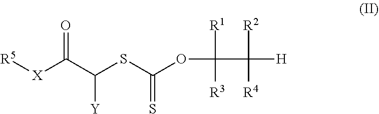
wherein:
R¹, R², R³ and R⁴, which are identical or different, represent a hydrogen atom or a group chosen from saturated or unsaturated heterocycloalkyl, alkyl, acyl, aryl, alkene, alkyne, cycloalkyl, or heterocycloaryl groups and polymer chains, it being understood that the radicals R¹, R², R³ and R⁴ may also form, together, a saturated, unsaturated or aromatic cycloalkyl or heterocycloalkyl group; it being understood that at least one of the radicals R¹ and R³ is other than a hydrogen atom;
Y has the same meaning as in the formula (I) above,
X represents NR²⁰, in which R²⁰ represents a hydrogen atom or an alkyl radical or —O—,
R⁵ is chosen from a saturated, unsaturated or aromatic heterocycloalkyl, alkyl, acyl, aryl, aralkyl or cycloalkyl group,
with a monomer comprising an ethylenic unsaturation of the following formula (III): 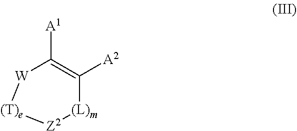
wherein:
A¹ and A², which are identical or different, represent a hydrogen atom or a fluorine atom,
L, m, T and e have the same meaning as in the formula (I) above;
wherein:
1) when A¹=A²=H and e=0, then W represents a hydrogen atom and Z² represents a group selected from the groups alkyl; aryl; P(O)(OR⁷)(OR^{7′}), in which the radicals R⁷ and R^{7′}, which are identical or different, represent a hydrogen atom or an alkyl radical; CₙF₂ₙ₊₁, in which n is an integer ranging from 1 to 20; SiR⁸ₚ(OR⁹)₃₋ₚ, in which the radicals R⁸ and R⁹, which are identical or different, represent a hydrogen atom or an alkyl radical and p is an integer equal to 0, 1 or 2; BF₃M⁺, in which M=K or Na; B(OR¹⁰)₂, in which the two radicals R¹⁰, which are identical or different, represent a hydrogen atom, an alkyl radical or form a carbon-based ring with the two oxygen atoms to which they are bonded; OR¹¹, in which R¹¹ represents a hydrogen atom or an alkyl, aryl or aralkyl radical; O(C═O)R¹², in which R¹² represents a hydrogen atom or an alkyl, aryl or aralkyl radical; O(C═O)OR¹³, in which R¹³ represents a hydrogen atom or an alkyl, aryl or aralkyl radical; N⁺R¹⁴R^{14′}R^{14″}A⁻, in which the radicals R¹⁴, R^{14′} and R^{14″}, which are identical or different, represent a hydrogen atom or an alkyl, aryl or aralkyl radical and A represents a chlorine or bromine atom; NR^{15′}(C═O)R¹⁵, in which the radicals R¹⁵ and R^{15′}, which are identical or different, represent a hydrogen atom or an alkyl or aryl radical or are connected together and form a ring such as a pyrrolidone or caprolactam ring; NR^{16′}(C═O)OR¹⁶, in which R¹⁶ and R^{16′}, which are identical or different, represent a hydrogen atom or an alkyl, aryl or aralkyl radical; CN; a halogen atom chosen from Cl, F, and Br; NCS; OCH₂-epoxy; COOR¹⁷, in which R¹⁷ represents a hydrogen atom, an alkyl, aryl or aralkyl radical; CONR¹⁸R^{18′}, in which R¹⁸ and R^{18′}, which are identical or different, represent a hydrogen atom or an alkyl or aryl radical; SO₂R¹⁹, in which R¹⁹ represents an alkyl or aryl radical; azide N₃, alkyne and C₂H₃;
2) when A¹=A²=H, m=1 and e=0, then W represents a hydrogen atom and Z² may also represent a hydrogen atom,
3) when A¹=A²=H, e=1 and m=0, then Z² and W are identical and represent —CH₂— or CO;
4) when A¹=A²=H, e=1, m=1 and T=CH₂, then Z²=W=—CH₂,
5) when A¹=A²=F, e=0 and m=0, then W represents a fluorine atom and Z²=F or represents a linear or branched chain OC_{q}F_{2q+1} in which q is an integer ranging from 1 to 5, [OCF₂CF(CF₃)]ᵣOC₃F₇, with r=an integer ranging from 0 to 20, OC₂F₄SO₂F, OCF₂CF(CF₃)OC₂F₄SO₂F, or OCF₂CF(CF₃)OC₂F₄CO₂CH₃; and
6) when A¹=A²=F, e=1 and m=0, then W=Z²=CF₂ and T=(CF₂)ₛ, with s=an integer ranging from 1 to 5;
7) when A¹=H, A²=F, and e=m=0, then W represents a hydrogen atom and Z²=F or CₙF₂ₙ₊₁, in which n is an integer ranging from 1 to 20;
to form a monoadduct of the following formula (IV): 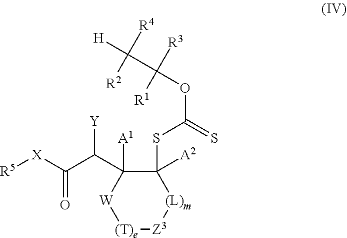
wherein:
A¹ and A², which are identical or different, represent a hydrogen atom or a fluorine atom,
R¹, R², R³, R⁴, R⁵, X and Y have the same meaning as in the formula (II) above,
L, m, T and e have the same meaning as in the formula (I) above,
wherein:
1) when A¹=A²=H and e=0, then W represents a hydrogen atom and Z³ represents a group selected from the groups alkyl; aryl; P(O)(OR⁷)(OR^{7′}), in which the radicals R⁷ and R^{7′}, which are identical or different, represent a hydrogen atom or an alkyl radical; CₙF₂ₙ₊₁, in which n is an integer ranging from 1 to 20; SiR⁸ₚ(OR⁹)₃₋ₚ, in which the radicals R⁸ and R⁹, which are identical or different, represent a hydrogen atom or an alkyl radical and p is an integer equal to 0, 1 or 2; BF₃M⁺, in which M=K or Na; B(OR¹⁰)₂, in which the two radicals R¹⁰, which are identical or different, represent a hydrogen atom, an alkyl radical or form a carbon-based ring with the two oxygen atoms to which they are bonded; OR¹¹, in which R¹¹ represents a hydrogen atom or an alkyl, aryl or aralkyl radical; O(C═O)R¹², in which R¹² represents a hydrogen atom or an alkyl, aryl or aralkyl radical; O(C═O)OR¹³, in which R¹³ represents a hydrogen atom or an alkyl, aryl or aralkyl radical; N⁺R¹⁴R^{14′}R^{14″}A⁻, in which the radicals R¹⁴, R^{14′} and R^{14″}, which are identical or different, represent a hydrogen atom or an alkyl, aryl or aralkyl radical and A represents a chlorine or bromine atom; NR^{15′}(C═O)R¹⁵, in which the radicals R¹⁵ and R^{15′}, which are identical or different, represent a hydrogen atom or an alkyl or aryl radical or are connected together and form a ring such as a pyrrolidone or caprolactam ring; NR^{16′}(C═O)OR¹⁶, in which R¹⁶ and R^{16′}, which are identical or different, represent a hydrogen atom or an alkyl, aryl or aralkyl radical; CN; a halogen atom chosen from Cl, F, and Br; NCS; OCH₂-epoxy; COOR¹⁷, in which R¹⁷ represents a hydrogen atom, an alkyl, aryl or aralkyl radical; CONR¹⁸R^{18′}, in which R¹⁸ and R^{18′}, which are identical or different, represent a hydrogen atom or an alkyl or aryl radical; SO₂R¹⁹, in which R¹⁹ represents an alkyl or aryl radical; azide N₃, alkyne, and a group of the following formula (V): 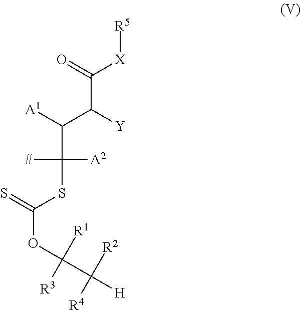
in which the symbol # is the point of attachment of the group of formula (V) to L and Y has the same meaning as that chosen for the radical Y of formula (IV),
2) when A¹=A²=H, m=1 and e=0, then W represents a hydrogen atom and Z³ may also represent a hydrogen atom,
3) when A¹=A²=H, e=1 and m=0, then Z³ and W are identical and represent —CH₂— or CO;
4) when A¹=A²=H, e=1, m=1 and T=CH₂, then Z³=W=—CH₂,
5) when A¹=A²=F, e=0 and m=0, then W represents a fluorine atom and Z³=F or represents a linear or branched chain OC_{q}F_{2q+1} in which q is an integer ranging from 1 to 5, [OCF₂CF(CF₃)]ᵣOC₃F₇, with r=an integer ranging from 0 to 20, OC₂F₄SO₂F, OCF₂CF(CF₃)OC₂F₄SO₂F, or OCF₂CF(CF₃)OC₂F₄CO₂CH₃; and
6) when A¹=A²=F, e=1 and m=0, then W=Z³=CF₂ and T=(CF₂)ₛ, with s=an integer ranging from 1 to 5;
7) when A¹=H, A²=F, and e=m=0, then W represents a hydrogen atom and Z³=F or CₙF₂ₙ₊₁, in which n is an integer ranging from 1 to 20;
then
2) a step of thermolysis of the monoadduct of formula (IV) obtained above in the preceding step, to form a corresponding substituted thiolactone of formula (I).

2. Process according to claim 1, wherein said process is carried out for the preparation of thiolactones of formula (I) in which:
Z¹ is a group chosen from the groups P(O)(OR⁷)(OR^{7′}); CₙF₂ₙ₊₁; B(OR¹⁰)₂; OR¹¹; SiR⁸ₚ(OR⁹)₃₋ₚ; NR^{15′}(C═O)R¹⁵, in which R^{15′} is a hydrogen atom and NR^{16′}(C═O)OR¹⁶, in which R^{16′} is a hydrogen atom, and/or
Y is a hydrogen atom or a group chosen from an alkyl radical.

3. Process according to claim 1, wherein said process is carried out for the preparation of substituted thiolactones of formula (I) in which:
Z¹ is a group chosen from the groups dimethylphosphonate and diethylphosphonate; CₙF₂ₙ₊₁; B(OR¹⁰)₂, OR¹¹, SiR⁸ₚ(OR⁹)₃₋ₚ, NR^{15′}(C═O)R¹⁵, in which R^{15′} is a hydrogen atom and NR^{16′}(C═O)OR¹⁶, in which R^{16′} is a hydrogen atom, and/or
Y is a hydrogen atom or a methyl or hydroxymethyl group.

4. Process according to claim 1, wherein said process leads to the formation of a thiolactone of formula (I), chosen from:
dimethyl 5-oxo-tetrahydrothiophen-2-ylphosphonate,
diethyl (4-methyl-5-oxo-tetrahydrothiophen-2-yl)methylphosphonate,
diethyl (5-oxo-tetrahydrothiophen-2-yl)methylphosphonate,
3-methyl-5-pentyl-dihydrothiophen-2(3H)-one,
5-pentyl-dihydrothiophen-2(3H)-one,
3-methyl-5-(perfluorooctyl)dihydrothiophen-2(3H)-one,
3-methyl-5-phenyldihydro-2H-thieno[2,3-c]pyrrole-2,4,6(3H, 5H)-trione,
3-methyl-5-(perfluorobutyl)dihydrothiophen-2(3H)-one,
(4-methyl-5-oxo-tetrahydrothiophen-2-yl)phosphonic acid,
((4-methyl-5-oxo-tetrahydrothiophen-2-yl)methyl)phosphonic acid,
(5-oxo-tetrahydrothiophen-2-yl)methylphosphonic acid,
3-methyl-5-(trimethoxysilyl)dihydrothiophen-2(3H)-one,
5-(trimethoxysilyl)dihydrothiophen-2(3H)-one,
tert-butyl-N-(4-methyl-5-oxo-tetrahydrothiophen-2-yl)carbamate,
tert-butyl (5-oxotetrahydrothiophen-2-yl)carbamate,
3-methyl-5-(oxiran-2-ylmethoxy)dihydrothiophen-2(2H)-one,
5-((oxiran-2-yloxy)methyl)dihydrothiophen-2(3H)-one,
3-methyl-5-(4,4,5,5-tetramethyl-1,3,2-dioxaborolan-2-yl)dihydrothiophen-2(3H)-one,
(5-oxo-tetrahydrothiophen-2-yl)phosphonic acid,
5-(4,4,5,5-tetramethyl-1,3,2-dioxaborolan-2-yl)dihydrothiophen-2(3H)-one,
5-(perfluorooctyl)dihydrothiophen-2(3H)-one,
5-(perfluorobutyl)dihydrothiophen-2(3H)-one,
dihydro-5-(3-(tetrahydro-4-methyl-5-oxothiophen-2-yl)propyl)-3-methylthiophen-2(3H)-one,
5-(9-hydroxynonyl)-3-methyldihydrothiophen-2(3H)-one,
5-(9-bromononyl)₃-methyldihydrothiophen-2(3H)-one,
5,5′-(ethane-1,2-diyl)bis(3-methyldihydrothiophen-2(3H)-one, and
5,5′-(hexane-1,6-diyl)bis(3-methyldihydrothiophen-2(3H)-one.

5. Process according to claim 1, wherein the step 1) of preparation of the monoadduct of formula (IV) is carried out without solvent, in water or in an organic solvent.

6. Process according to claim 1, wherein the radical initiator used during step 1) is chosen from organic peroxides, azo derivatives, redox couples that generate radicals and redox systems.

7. Process according to claim 6, wherein the organic peroxides are chosen from dilauroyl peroxide, t-butyl peroxyacetate, t-butyl peroxybenzoate, t-butyl peroxyoctoate, t-butyl peroxydodecanoate, t-butyl peroxyisobutyrate, t-amyl peroxypivalate, t-butyl peroxypivalate, diisopropyl peroxydicarbonate, dicyclohexyl peroxydicarbonate, dicumyl peroxide, dibenzoyl peroxide, potassium peroxydisulfate, sodium peroxydisulfate and ammonium peroxydisulfate.

8. Process according to claim 1, wherein the linker arm L is a linear alkyl chain, possibly interrupted by one or more heteroatoms, said hydrocarbon-based chain having from 1 to 100 carbon atoms.

9. Process according to claim 1, wherein the monomers of formula (III) are alkenes chosen from ethylene, propylene, 1-butene, 1-pentene, 1-hexene, 1-heptene, 1-octene, 1-nonene, 1-decene, perfluorohexylethylene and perfluorooctylethylene.

10. Process according to claim 1, wherein the monomers of formula (III) are allylic compounds chosen from allylic alcohol, N-allyl benzamide, ethyl N-allyl carbamate, tert-butyl N-allyl carbamate, 2-allyl-4,4,5,5-tetramethyl-1,3,2-dioxaborolane, 2-allyl-6-methyl-1,3,6,2-dioxazaborocane-4,8-dione, allylboronic acid, diethyl allylphosphonate, allyl phosphonic dichloride, dimethyl allylphosphonate, allyl cyanide, allyl isothiocyanate, allyl glycidyl ether, allyl benzyl ether, allyl phenyl ether, allyl butyl ether, allyl ethyl ether, allyl methylsulfone, allyl phenylsulfone, allyl chloride, allyl bromide and allyl fluoride.

11. Process according to claim 1, wherein step 1) is carried out at a temperature varying from 10 to 140° C.

12. Process according to claim 1, wherein step 2) of thermolysis is carried out without solvent.

13. Process according to claim 1, wherein at least one of the groups R¹ or R³ is other than a hydrogen atom.

14. Substituted thiolactones of the following formula (I′): 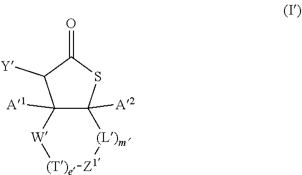
wherein:
A′¹, A′², Y′, Z′, L′, m′, T′ and e′ may respectively assume the same meanings as those defined in claim 1 for A¹, A², Y, Z¹, L, m, T and e for the thiolactones of formula (I), with the proviso that:
when e′=0 and W′=H and m′=0 and Y′=hydrogen, then Z^{1′} is other than a hydrogen atom, than a linear alkyl chain or than a phenyl ring; and
when e′=0 and W′=H and m′=0 and Y′ is a substituent having a nitrogen atom directly bonded to the thiolactone ring, then Z^{1′} is other than a hydrogen atom.

15. Substituted thiolactones of formula (I′) according to claim 14, wherein they are chosen from:
dimethyl 5-oxo-tetrahydrothiophen-2-ylphosphonate,
diethyl (4-methyl-5-oxo-tetrahydrothiophen-2-yl)methylphosphonate,
diethyl (5-oxo-tetrahydrothiophen-2-yl)methylphosphonate,
3-methyl-5-pentyl-dihydrothiophen-2(3H)-one,
5-pentyl-dihydrothiophen-2(3H)-one,
3-methyl-5-(perfluorooctyl)dihydrothiophen-2(3H)-one,
3-methyl-5-phenyldihydro-2H-thieno[2,3-c]pyrrole-2,4,6(3H, 5H)-trione,
3-methyl-5-(perfluorobutyl)dihydrothiophen-2(3H)-one,
(4-methyl-5-oxo-tetrahydrothiophen-2-yl)phosphonic acid,
((4-methyl-5-oxo-tetrahydrothiophen-2-yl)methyl)phosphonic acid,
(5-oxo-tetrahydrothiophen-2-yl)methylphosphonic acid,
3-methyl-5-(trimethoxysilyl)dihydrothiophen-2(3H)-one,
5-(trimethoxysilyl)dihydrothiophen-2(3H)-one,
tert-butyl-N-(4-methyl-5-oxo-tetrahydrothiophen-2-yl)carbamate,
tert-butyl (5-oxotetrahydrothiophen-2-yl)carbamate,
3-methyl-5-(oxiran-2-ylmethoxy)dihydrothiophen-2(2H)-one,
5-((oxiran-2-yloxy)methyl)dihydrothiophen-2(3H)-one,
3-methyl-5-(4,4,5,5-tetramethyl-1,3,2-dioxaborolan-2-yl)dihydrothiophen-2(3H)-one,
(5-oxo-tetrahydrothiophen-2-yl)phosphonic acid,
5-(4,4,5,5-tetramethyl-1,3,2-dioxaborolan-2-yl)dihydrothiophen-2(3H)-one,
5-(perfluorooctyl)dihydrothiophen-2(3H)-one,
5-(perfluorobutyl)dihydrothiophen-2(3H)-one,
dihydro-5-(3-(tetrahydro-4-methyl-5-oxothiophen-2-yl)propyl)-3-methylthiophen-2(3H)-one,
5-(9-hydroxynonyl)-3-methyldihydrothiophen-2(3H)-one,
5-(9-bromononyl)₃-methyldihydrothiophen-2(3H)-one,
5,5′-(ethane-1,2-diyl)bis(3-methyldihydrothiophen-2(3H)-one, and
5,5′-(hexane-1,6-diyl)bis(3-methyldihydrothiophen-2(3H)-one.

16. At least one substituted thiolactone of formula (I) obtained according to the process as defined in claim 1, said at least one substituted thiolactone configured for the synthesis of polymers or for surface functionalization or polymer functionalization.

17. least one thiolactone of formula (I′) as defined in claim 14, said at least one substituted thiolactone configured for the synthesis of polymers or for surface functionalization or polymer functionalization.
